(19) United States
(12) Patent Application Publication
(10) Pub. No.: US 2025/0333484 A1
(43) Pub. Date: Oct. 30, 2025

(54) BISPECIFIC ANTIBODIES TO EBOLA VIRUS GLYCOPROTEIN AND THEIR USE

(71) Applicant: The U.S.A., as represented by the Secretary, Department of Health and Human Services, Bethesda, MD (US)

(72) Inventors: Nancy J. Sullivan, Brookline, MA (US); John N. Misasi, Boston, MA (US); Ke Bai, Potomac, MD (US); Mangaiarkarasi Asokan, Madurai (IN); Kendra Leigh, Cambridge (GB); Amarendra Pegu, Potomac, MD (US); John R. Mascola, Rockville, MD (US); Megan E. DeMouth, New York, NY (US); Christopher D. Stringham, Randolph, NJ (US); Olamide K. Oloniniyi, Bethesda, MD (US)

(73) Assignee: The U.S.A., as represented by the Secretary, Department of Health and Human Services, Bethesda, MD (US)

(21) Appl. No.: 18/855,271

(22) PCT Filed: Apr. 14, 2023
(86) PCT No.: PCT/US2023/065775

§ 371 Date: Oct. 8, 2024

Related U.S. Application Data
(60) Provisional application No. 63/330,877, filed on Apr. 14, 2022.

(51) Int. Cl. C07K 16/10; A61K 39/00; A61P 37/04

(52) U.S. Cl. CPC C07K 16/10; A61P 37/04; A61K 2039/505; C07K 2317/31; C07K 2317/76; C07K 2317/92

(57) ABSTRACT

A bispecific monoclonal antibody that specifically binds two distinct epitopes of the Ebola virus (EBOV) glycoprotein (GP) is described. The bispecific antibody is comprised of the antigen binding domains of GP-specific monoclonal antibodies mAb114 and S1-4-A09 (“A09”). The EBOV GP bispecific monoclonal antibody (mAb114×A09 or BiSp107) exhibits synergistic neutralization of pseudotyped virus expressing EBOV GP compared with the neutralization capacity of the combination of the individual parental antibodies. Methods for pre- and post-exposure prophylaxis and treatment are described.

### CROSS REFERENCE TO RELATED APPLICATIONS

This application claims the benefit of U.S. Provisional Application No. 63/330,877, filed Apr. 14, 2022, which is herein incorporated by reference in its entirety.

### FIELD

This disclosure concerns bispecific monoclonal antibodies that specifically bind two different epitopes of Ebola virus (EBOV) glycoprotein and their use for pre- and post-exposure prophylaxis and treatment of EBOV infection.

### INCORPORATION OF ELECTRONIC SEQUENCE LISTING

The electronic sequence listing, submitted herewith as an XML file named 4239-106252-02.xml (29,458 bytes), created on Apr. 4, 2023, is herein incorporated by reference in its entirety.

### BACKGROUND

Ebola virus (EBOV) caused a global public health epidemic with more than 28,616 cases in West Africa between 2014 and 2016 and 3470 cases in Kivu of Democratic Republic of the Congo (DRC). EBOV causes severe illness and is associated with a high mortality rate. EBOV initially targets macrophages and dendritic cells via micropinocytosis from the enveloped glycoprotein (GP), then it is taken into the low-pH compartment of proteasomes and lysosomes. In these compartments, the EBOV GP is cleaved by cysteine proteases (cathepsin B and L). As a result, the heavily glycosylated region of GP is cleaved off, leaving a minimal GPI core, exposing the receptor-binding domain for Niemann-Pick C1 protein (NPC1). In a randomized controlled trial in participants with confirmed EBOV infection, a single, intravenous administration of 50 mg/kg mAb114 reduced mortality significantly. Ansuvimab (mAb114) was approved by the U.S. Food and Drug Administration (FDA) as a licensed treatment against Zaire ebolavirus in December 2020. Despite decreasing mortality, monotherapy in non-human primates with mAb114 results in a transient viremia. Thus, a need remains for additional EBOV therapeutics effective for both pre-exposure and post-exposure prophylaxis, as well as for the treatment of EBOV infection without transient viremia. Furthermore, current treatments have lower efficacy in human trials when patients present with severe disease, indicating a need for antibody treatments with improved properties. No therapeutic has been shown to protect against death from Ebola virus disease (EVD) when given prior to virus exposure. In view of this, current treatments cannot be used for pre-exposure prophylaxis. Thus, a need remains for additional EBOV therapeutics that are effective for both pre-exposure prophylaxis and post-exposure treatment.

### SUMMARY

A bispecific monoclonal antibody that specifically binds two different epitopes of the EBOV glycoprotein (GP) is described. A first antigen binding portion of the bispecific monoclonal antibody is derived from mAb114, which binds an epitope within the chalice of GP, distal to the virus envelope. A second antigen binding portion of the bispecific antibody is derived from antibody S1-4-A09 (“A09”), which binds an epitope at the base of the GP trimer, contacting GP1 and GP2 of one promoter (see FIGS. 4A-4C). The disclosed EBOV GP-bispecific monoclonal antibody, referred to as mAb114×A09 or BiSp107, exhibits synergistic neutralization of pseudotyped virus expressing EBOV GP compared with the neutralization capacity of the combination of the individual parental antibodies (mAb114+A09), significantly reduces the risk of virus escape, and protects against lethal EBOV challenge when administered either post-infection or pre-exposure.

Provided herein is a bispecific monoclonal antibody that specifically binds two distinct epitopes of the EBOV GP. In some aspects, the first epitope is within the chalice of the GP receptor binding domain (RBD) and the second epitope is at the base of the GP trimer (see FIGS. 4B-4C). In some aspects, the bispecific monoclonal antibody includes a first antigen binding portion that includes a heavy chain variable domain and a light chain variable domain, wherein the heavy chain variable domain and light chain variable domain complementarity determining regions (CDRs) are from the GP-specific monoclonal antibody mAb114; and a second antigen binding portion that includes a heavy chain variable domain and a light chain variable domain, wherein the heavy chain variable domain and light chain variable domain CDRs are from the GP-specific antibody A09.

Compositions that include a disclosed bispecific monoclonal antibody and a pharmaceutically acceptable carrier are also provided.

Further provided are nucleic acid molecules encoding a disclosed bispecific monoclonal antibody, or a portion thereof, such as a heavy chain or a light chain. Expression vectors that include a disclosed nucleic acid molecule, as well as host cells that include a nucleic acid molecule or vector disclosed herein are also provided.

Also provided is a method of producing a bispecific monoclonal antibody that specifically binds EBOV GP. In some aspects, the method includes transfecting host cells with a first expression vector comprising a nucleotide sequence encoding the mAb114 heavy chain (e.g., the nucleotide sequence of SEQ ID NO: 1), a second expression vector comprising a nucleotide sequence encoding the mAb114 light chain (e.g., the nucleotide sequence of SEQ ID NO: 3), a third expression vector comprising a nucleotide sequence encoding the A09 heavy chain (e.g., the nucleotide sequence of SEQ ID NO: 5) and a fourth expression vector comprising a nucleotide sequence encoding the A09 light chain (e.g., the nucleotide sequence of SEQ ID NO: 7); and purifying the bispecific monoclonal antibody from the host cells and/or host cell culture supernatant.

Further provided are methods of preventing, inhibiting or treating an EBOV infection in a subject. In some aspects, the method includes administering to the subject a therapeutically effective amount of an EBOV GP bispecific monoclonal antibody or composition disclosed herein. The method can include post-infection treatment, post-exposure prophylaxis or pre-exposure prophylaxis.

The foregoing and other objects and features of the disclosure will become more apparent from the following detailed description, which proceeds with reference to the accompanying figures.

### BRIEF DESCRIPTION OF THE DRAWINGS

FIGS. 1A-1C: Design of bispecific BiSp107 antibody. (FIG. 1A) Schematic of CrossMab design of BiSp107. (FIGS. 1B-1C) Class averages of single particles from negative-stain electron micrographs of Fab114 and FabA09 from parental Fab (FIG. 1B) and bispecific mAb (FIG. 1C).

FIGS. 2A-2C: Binding characteristics of BiSp107. (FIG. 2A) Bispecificity of BiSp107. Simultaneous binding of each arm of bispecific IgG was demonstrated by sandwich assay using biolayer interferometry (BLI). Octet sensor was loaded with probe against one arm and then probed sequentially with bispecific IgG and the probe against the second arm. As controls, parental IgGs were used in place of the bispecific IgG. (FIG. 2B) Kinetics of binding as determined by BLI. Kinetics of Fab114 (left panel) or mAb114 arm of BiSp107 (right panel) binding to GP N-terminus probe. (FIG. 2C) Saturation binding kinetics of BiSp107 and parental mAbs to GP full probe using BLI. n=3 replicates.

FIGS. 3A-3D: Functional characterization of BiSp107. (FIG. 3A) Cathepsin cleavage blocking analysis. GP ΔMuc was incubated with the indicated antibodies followed by cleavage at pH 5.5 by Cat L at 37° C. Samples were removed at indicated intervals and analyzed by immunoblot for GP1. n=3 replicates; representative image shown. (FIG. 3B) Percent inhibition of NPC1 domain C (NPC1-dC) binding by antibodies (mAb114, mAbA09, KZ52, 13C6, mAb114+mAbA09, BiSp107, or NPC1-dC or VRC-01) to thermolysin cleaved GP (GP_{THL}) as measured by BLI. Dashed line represents 100% inhibition of binding. n=3 replicates; representative experiment shown. (FIG. 3C) Pseudotyped EBOV GP lentivirus particles were incubated with increasing amounts of purified mAbs and used to infect HEK293T cells. Percent infectivity=[(RLU with antibody)/(RLU without antibody)]×100%, mean+SD (n=3, representative experiment shown). (FIG. 3D) Pseudotyped EBOVGP VSV particles were incubated with increasing concentrations (5.1×10⁻⁶ to 50 μg/mL) of either parental mAb114 or mAbA09 antibodies and combinations of the mAb114+mAbA09 and BiSp107. Every day, wells were assessed for CPE and the highest concentration well with >20% CPE was passaged forward onto fresh cells and antibody containing media. Shown is the maximum concentration with >20% CPE for each of the test conditions in each round of selection. Once 50 μg/mL was reached, virus was no longer passaged forward.

FIGS. 4A-4C: Entry mechanism of Ebola showing the distinct biochemical steps targeted by mAb114 and mAbA09. (FIG. 4A) Entry mechanism of Ebola virus. (FIGS. 4B-4C) Distinct biochemical steps targeted by mAb114 (FIG. 4B) and mAbA09 (FIG. 4C).

FIGS. 5A-5D: Characterization of bispecific antibodies. (FIG. 5A) A table listing parental antibodies. (FIG. 5B) A table listing bispecific antibodies made in the CH1-CL format. (FIGS. 5C-5D) Bispecific antibodies were expressed in Expi293 cells and purified mAbs were characterized by SDS-PAGE (FIG. 5C) and mass spectrometry (FIG. 5D).

FIGS. 6A-6B: Kinetics of binding as determined by biolayer interferometry. (FIG. 6A) Kinetics readout of binding. (FIG. 6B) Kinetics of Fab A09 (left panel) or A09 arm of BiSp107 (right panel) binding to GP full probe pre-saturated with mAb114 to block mAb114 binding site.

FIGS. 7A-7B: Pharmacokinetic (PK) study in human FcRn transgenic mice. (FIG. 7A) Naïve human FcRn mice were infused with 5 mg/kg of mAb114, mAbA09 or BiSp107 (n=5 for each antibody) and serum antibody concentrations were measured by an anti-idiotype based ELISA. Error bars represent SEM. (FIG. 7B) Calculated PK parameters for the parental mAbs and BiSp107.

FIGS. 8A-8F: Passive transfer of BiSp107 and protection in NHPs. (FIG. 8A) Schematic of the study design. Animals were challenged with a lethal dose of EBOV on day 0 and given injections of antibody totaling 50 mg/kg post-exposure (three doses on days 1, 2 and 3, or a single dose on day 4) or pre-exposure (three doses on days −3, −2, −1 or a single dose on day −3). (FIG. 8B) The percent of NHP protected from death in each treatment group. (FIGS. 8C-8F) For the indicated treatment groups, plasma viremia as measured by quantitative reverse transcription PCR (qRT-PCR) is presented in genome equivalents per milliliter (ge/ml), the percent of lymphocytes in blood and concentrations of ALT (alanine transaminase) and CRE (creatinine) in plasma samples are shown.

FIGS. 9A-9D: Hematologic and chemistry data. (FIGS. 9A, 9C) Ebola GP-specific ELISA titer (reciprocal EC90), post-exposure (FIG. 9A) and pre-exposure (FIG. 9C). (FIGS. 9B, 9D) Selected hematologic and chemistry data post-exposure (FIG. 9B) and pre-exposure (FIG. 9D). PLT, platelets; ALT, alanine transaminase; CRE, creatinine.

### SEQUENCE LISTING

The nucleic and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is a nucleic acid sequence encoding the mAb114 heavy chain.

SEQ ID NO: 2 is an amino acid sequence of the mAb114 heavy chain.

SEQ ID NO: 3 is a nucleic acid sequence encoding the mAb114 light chain.

SEQ ID NO: 4 is an amino acid sequence of the mAb114 light chain.

SEQ ID NO: 5 is a nucleic acid sequence encoding the A09 heavy chain.

SEQ ID NO: 6 is an amino acid sequence of the A09 heavy chain.

SEQ ID NO: 7 is a nucleic acid sequence encoding the A09 light chain.

SEQ ID NO: 8 is an amino acid sequence of the A09 light chain.

SEQ ID NO: 9 is an amino acid sequence of a GCN4 site followed by Avitag peptide and His tags.

SEQ ID NOS: 10 and 11 are PCR primer sequences.

SEQ ID NOs: 12-17 are the amino acid sequences of the mAb114 CDRs.

SEQ ID NOs: 18-23 are the amino acid sequences of the A09 CDRs.

### DETAILED DESCRIPTION

Several neutralizing mAb or mAb cocktails that bind to EBOV GP have been previously discovered, including ZMapp, MIL77E, mAb114 and REGN-EB3. A randomized, controlled trial of three antibody treatments, ZMapp, mAb114, REGN-EB3, and the anti-viral Remdesivir, was conducted in the Democratic Republic of the Congo (DRC) as a part of the emergency response to the 2018 EBOV outbreak. The reported data showed that treatment with mAb114 reduced the mortality rate from 67% to 34% (52/155). However, since monoclonal antibody treatments are prone to encounter virus escape mutants, the studies disclosed herein sought to construct a bispecific antibody with improved therapeutic features, including enhanced neutralization characteristics, delayed mutant escape, and the ability to provide pre-exposure prophylaxis and post-exposure protection against Ebola virus disease. In the present disclosure, a bispecific antibody (BiSp107) containing the antigen binding arms of two different EBOV neutralizing antibodies (mAb114 and S1-4-A09), was constructed. The ability of this bispecific IgG to recognize two different binding epitopes of EBOV GP has contributed to superior binding kinetics, neutralization potency, and the capacity to prevent the generation of escape mutations in an in vitro assay system. Moreover, using the NHP platform, BiSp107 demonstrated full protection when given after infection or when given as pre-exposure prophylaxis with single or multiple doses.

### I. Abbreviations

ALT alanine transaminase
BLI biolayer interferometry
CDR complementarity determining region
CH constant heavy
CL constant light
CRE creatinine
EBOV Ebola virus
EVD Ebola virus disease
FcRn neonatal Fc receptor
GP glycoprotein
GPΔMuc mucin-like domain deleted glycoprotein
HC heavy chain
LC light chain
NHP non-human primate
NPC1 Niemann-Pick C1 protein
PK pharmacokinetics
VH variable heavy
VL variable light

### II. Summary of Terms

Unless otherwise noted, technical terms are used according to conventional usage. Definitions of many common terms in molecular biology may be found in Krebs et al. (eds.), Lewin's genes XII, published by Jones & Bartlett Learning. 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Administration: The introduction of a composition into a subject by a chosen route. Administration can be local or systemic. For example, if the chosen route is intravenous, the composition is administered by introducing the composition into a vein of the subject. Exemplary routes of administration include, but are not limited to, oral, injection (such as subcutancous, intramuscular, intradermal, intraperitoneal, and intravenous), infusion, sublingual, rectal, transdermal (for example, topical), intranasal, vaginal, and inhalation routes.

Antibody: An immunoglobulin, antigen-binding fragment, or derivative thereof, that specifically binds and recognizes an analyte (antigen) such as Ebola virus GP. The term “antibody” is used herein in the broadest sense and encompasses various antibody structures, including but not limited to monoclonal antibodies, polyclonal antibodies, multispecific antibodies (e.g., bispecific antibodies), and antibody fragments, so long as they exhibit the desired antigen-binding activity.

Non-limiting examples of antibodies include, for example, intact immunoglobulins and variants and fragments thereof known in the art that retain binding affinity for the antigen. Examples of antibody fragments include but are not limited to Fv, Fab, Fab′, Fab′-SH, F(ab′)₂; diabodies; linear antibodies; single-chain antibody molecules (e.g., scFv, VHH); and multispecific antibodies formed from antibody fragments. Antibody fragments include antigen binding fragments either produced by the modification of whole antibodies or those synthesized de novo using recombinant DNA methodologies (see, e.g., Kontermann and Dübel (Eds.), Antibody Engineering, Vols. 1-2, 2^{nd} ed., Springer-Verlag, 2010).

A single-chain antibody (scFv) is a genetically engineered molecule containing the V_{H} and V_{L} domains of one or more antibody(ies) linked by a suitable polypeptide linker as a genetically fused single chain molecule (see, for example, Bird et al., Science, 242(4877):423-426, 1988; Huston et al., Proc. Natl. Acad. Sci. U.S.A., 85(16):5879-5883, 1988; Ahmad et al., Clin. Dev. Immunol., 2012, doi:10.1155/2012/980250; Marbry and Snavely, IDrugs, 13(8):543-549, 2010). The intramolecular orientation of the V_{H}-domain and the V_{L}-domain in a scFv, is typically not decisive for scFvs. Thus, scFvs with both possible arrangements (V_{H}-domain-linker domain-V_{L}-domain; V_{L}-domain-linker domain-V_{H}-domain) may be used.

In a dsFv the V_{H} and V_{L} have been mutated to introduce a disulfide bond to stabilize the association of the chains. Diabodies also are included, which are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci. U.S.A., 90(14):6444-6448, 1993; Poljak et al., Structure, 2(12):1121-1123, 1994).

Antibodies also include genetically engineered forms such as chimeric antibodies (such as humanized murine antibodies) and heteroconjugate antibodies (such as bispecific antibodies).

Typically, a naturally occurring immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain). In several aspects, the V_{H} and V_{L} combine to specifically bind the antigen. In additional aspects, only the V_{H} is required. For example, naturally occurring camelid antibodies consisting of a heavy chain only (VHH) are functional and stable in the absence of light chain. Any of the disclosed antibodies can include a heterologous constant domain. For example, the antibody can include constant domain that is different from a native constant domain, such as a constant domain including one or more modifications (such as the “LS” mutations) to increase half-life.

References to “V_{H}” or “VH” refer to the variable region of an antibody heavy chain, including that of an antigen binding fragment, such as Fv, scFv, dsFv or Fab. References to “V_{L}” or “VL” refer to the variable domain of an antibody light chain, including that of an Fv, scFv, dsFv or Fab.

The V_{H} and V_{L} contain a “framework” region interrupted by three hypervariable regions, also called “complementarity-determining regions” or “CDRs” (see, e.g., Kabat et al., Sequences of Proteins of Immunological Interest, 5^{th} ed., NIH Publication No. 91-3242, Public Health Service, National Institutes of Health, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (Sequences of Proteins of Immunological Interest, 5^{th} ed., NIH Publication No. 91-3242, Public Health Service, National Institutes of Health, U.S. Department of Health and Human Services, 1991; “Kabat” numbering scheme), Al-Lazikani et al., (“Standard conformations for the canonical structures of immunoglobulins,” J. Mol. Bio., 273(4):927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27(1):55-77, 2003; “IMGT” numbering scheme). The CDRs of each chain are typically referred to as CDR1, CDR2, and CDR3 (from the N-terminus to C-terminus) and are also typically identified by the chain in which the particular CDR is located. Thus, a V_{H} CDR3 is the CDR3 from the V_{H} of the antibody in which it is found, whereas a V_{L} CDR1 is the CDR1 from the V_{L} of the antibody in which it is found. Light chain CDRs are sometimes referred to as LCDR1, LCDR2, and LCDR3. Heavy chain CDRs are sometimes referred to as HCDR1, HCDR2, and HCDR3.

A “monoclonal antibody” is an antibody obtained from a population of substantially homogeneous antibodies, that is, the individual antibodies comprising the population are identical and/or bind the same epitope, except for possible variant antibodies, for example, containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples, monoclonal antibodies are isolated from a subject. Monoclonal antibodies can have conservative amino acid substitutions which have substantially no effect on antigen binding or other immunoglobulin functions. (See, for example, Greenfield (Ed.), Antibodies: A Laboratory Manual, 2^{nd} ed. New York: Cold Spring Harbor Laboratory Press, 2014.)

A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one aspect, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some aspects, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1^{st} ed. New York: Cold Spring Harbor Laboratory Press, 2004; Lonberg, Nat. Biotechnol., 23(9):1117-1125, 2005; Lonberg, Curr. Opin. Immunol. 20(4):450-459, 2008).

Binding affinity: Affinity of an antibody (or bispecific antibody) for an antigen. In one aspect, affinity is calculated by a modification of the Scatchard method described by Frankel et al., Mol. Immunol., 16:101-106, 1979. In another aspect, binding affinity is measured by an antigen/antibody dissociation rate. In another aspect, a high binding affinity is measured by a competition radioimmunoassay. In another aspect, binding affinity is measured by ELISA. In some aspects, binding affinity is measured using the Octet system (Creative Biolabs), which is based on bio-layer interferometry (BLI) technology. In other aspects, Kd is measured using surface plasmon resonance assays using a BIACORES-2000 or a BIACORES-3000 (BIAcore, Inc., Piscataway, N.J.). In other aspects, antibody affinity is measured by flow cytometry or by surface plasmon reference. An antibody that “specifically binds” an antigen (such as EBOV glycoprotein) is an antibody that binds the antigen with high affinity and does not significantly bind other unrelated antigens.

Biological sample: A sample obtained from a subject. Biological samples include all clinical samples useful for detection of disease or infection (for example, EBOV infection) in subjects, including, but not limited to, cells, tissues, and bodily fluids, such as blood, derivatives and fractions of blood (such as serum), cerebrospinal fluid; as well as biopsied or surgically removed tissue, for example tissues that are unfixed, frozen, or fixed in formalin or paraffin. In a particular example, a biological sample is obtained from a subject having or suspected of having an EBOV infection.

Bispecific antibody: A recombinant molecule composed of two different antigen binding portions that consequently binds to two different antigenic epitopes. Bispecific antibodies include chemically or genetically linked molecules of two antigen-binding domains. The antigen binding domains can be linked using a linker. The antigen binding domains can be monoclonal antibodies, antigen-binding fragments (e.g., Fab, scFv), or combinations thereof. A bispecific antibody can include one or more constant domains, but does not necessarily include a constant domain.

Conjugate: A complex of two molecules linked together, for example, linked together by a covalent bond. In one aspect, a bispecific antibody disclosed herein is linked to an effector molecule, such as covalently linked to an effector molecule. The linkage can be by chemical or recombinant means. In one aspect, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because conjugates can be prepared from two molecules with separate functionalities, such as an antibody and an effector molecule, they are also sometimes referred to as “chimeric molecules.”

Conservative amino acid substitution: “Conservative” amino acid substitutions are those substitutions that do not substantially affect a function of a protein, such as the ability of the protein to interact with a target protein.

In some aspects, a conservative amino acid substitution in a bispecific EBOV GP-specific antibody is one that does not reduce binding of the bispecific antibody to EBOV GP by more than 10% (such as by more than 5%) compared to the EBOV GP binding of the corresponding antibody lacking the conservative amino acid substitution. In some aspects, the EBOV GP-specific antibody can include up to 1, 2, 3, 4, 5, 6, 7, 8, 9, or up to 10 conservative substitutions compared to a reference antibody and retain specific binding activity for GP, and/or EBOV neutralization activity.

Typically, individual substitutions, deletions or additions which alter, add or delete a single amino acid or a small percentage of amino acids (for instance less than 5%, in some aspects less than 1%) in an encoded sequence are conservative variations where the alterations result in the substitution of an amino acid with a chemically similar amino acid. The following six groups are examples of amino acids that are considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Degenerate variant: In the context of the present disclosure, a “degenerate variant” refers to a polynucleotide encoding a protein (for example, a bispecific antibody or portion thereof (such as a variable region) that specifically binds EBOV GP) that comprises a sequence that is degenerate as a result of the genetic code. There are twenty natural amino acids, most of which are specified by more than one codon. Therefore, all degenerate nucleotide sequences are included as long as the amino acid sequence of the bispecific antibody that binds EBOV GP encoded by the nucleotide sequence is unchanged.

Ebola virus (EBOV): A genus of enveloped, non-segmented, negative-sense, single-stranded RNA viruses that causes Ebola virus disease (EVD), formerly known as Ebola hemorrhagic fever (EHF), in humans. Ebola viruses spread through human-to-human transmission, with infection resulting from direct contact with blood, secretions, organs or other bodily fluids of infected people, and indirect contact with environments contaminated by such fluids.

The symptoms of EBOV infection and EVD are well-known. Briefly, in humans, Ebola viruses have an initial incubation period of 2 to 21 days (7 days on average, depending on the EBOV species) followed by rapid onset of non-specific symptoms such as fever, extreme fatigue, gastrointestinal complaints, abdominal pain, anorexia, headache, myalgias and/or arthralgias. These initial symptoms last for about 2 to 7 days after which more severe symptoms related to hemorrhagic fever occur, including hemorrhagic rash, epistaxis, mucosal bleeding, hematuria, hemoptysis, hematemesis, melena, conjunctival hemorrhage, tachypnea, confusion, somnolence, and hearing loss. In general, the symptoms last for about 7 to 14 days after which recovery may occur. Death can occur 6 to 16 days after the onset of symptoms. People are infectious as long as their blood and secretions contain the virus, which in some instances can be more than 60 days.

Immunoglobulin M (IgM) antibodies to the virus appear 2 to 9 days after infection whereas immunoglobulin G (IgG) antibodies appear approximately 17 to 25 days after infection, which coincides with the recovery phase. In survivors of EVD, both humoral and cellular immunity are detected, however, their relative contribution to protection is unknown.

Six distinct species of Ebolavirus are known, including Zaire ebolavirus, Bundibugyo ebolavirus, Reston ebolavirus, Sudan ebolavirus, Taï Forest ebolavirus, and Bombali ebolavirus. Bundibugyo ebolavirus, Sudan ebolavirus, and Zaire ebolavirus have been associated with large outbreaks of EVD in Africa and reported case fatality rates of up to 90%.

The EBOV genome includes about 19 kb, which encode seven structural proteins including NP (a nucleoprotein), VP35 (a polymerase cofactor), VP30 (a transcriptional activator), VP24, L (an RNA polymerase), and GP (a glycoprotein).

Ebola virus glycoprotein (GP): The virion-associated transmembrane glycoprotein of EBOV is initially synthesized as a precursor protein of about 676 amino acids in size, designated GP₀. Individual GP₀ polypeptides form a homotrimer and undergo glycosylation and processing to remove the signal peptide, as well as cleavage by a cellular protease between approximately positions 501/502 (from the initiating methionine) to generate separate GP₁ and GP₂ polypeptide chains, which remain associated via disulfide bonds as GP₁/GP₂ protomers within the homotrimer. The extracellular GP₁ trimer (approximately 153 kDa) is derived from the amino-terminal portion of the GP₀ precursors, and the GP₂ trimer (approximately 59 kDa), which includes extracellular, transmembrane, and cytosolic domains, is derived from the carboxyl-terminal portion of the GP₀ precursors. GP₁ is responsible for attachment to new host cells while GP₂ mediates fusion with those cells. GP₁ contains a mucin-like domain from position 309-501 that is dispensable for infection. Given this, the domain is often removed in order to more efficiently produce viruses and proteins for assays and is referred to as GPΔMuc.

A variant transcript of the gene encoding EBOV GP encodes a soluble glycoprotein (sGP) that is secreted from the viral host cell. The transcript for sGP is created via stuttering of the polymerase on a slippery sequence composed of 7U's resulting in either transcript with 7A's, which codes for sGP, or 8A's, which codes for GP. sGP and GP₁ are identical in their first 295 N-terminal amino acids, whereas the remaining 69 C-terminal amino acids of sGP and 206 amino acids of GP₁ are encoded by different reading frames. It has been suggested that secreted sGP may effectively bind antibodies that might otherwise be protective (see, e.g., Sanchez et al., Proc. Natl. Acad. Sci. U.S.A., 93(8): 3602-3607, 1996; and Volchkov et al., Virology. 245(1): 110-119, 1998).

Comparisons of the predicted amino acid sequences for the GPs of the different species of EBOV show conservation of amino acids in the amino-terminal and carboxy-terminal regions with a highly variable region in the middle of the protein (Sanchez et al., Virus Res. 29(3): 215-240, 1993; Sanchez et al. Proc. Natl. Acad. Sci. U.S.A., 93(8): 3602-3607, 1996). The GPs of the Ebola viruses are highly glycosylated and contain both N-linked and O-linked carbohydrates that contribute up to 50% of the molecular weight of the protein. Most of the glycosylation sites are found in the central variable region of GP.

Effective amount (or therapeutically effective amount): A quantity of a specific substance sufficient to achieve a desired effect in a subject to whom the substance is administered. For instance, this can be the amount necessary to inhibit, prevent or treat an EBOV infection, or to measurably alter outward symptoms of the infection.

In some aspects, a therapeutically effective amount of a disclosed bispecific antibody that binds to EBOV GP is an amount necessary to reduce or inhibit an EBOV infection (for example, as measured by infection of cells, or by number or percentage of subjects infected by EBOV, or by an increase in the survival time of infected subjects, or by reduction in symptoms associated with EBOV infection) by a desired amount, for example by at least 10%, at least 20%, at least 50%, at least 60%, at least 70%, at least 80%, at least 90%, at least 95%, at least 98%, or even at least 100% (elimination or prevention of detectable EBOV infection), as compared to a suitable control.

The effective amount (or therapeutically effective amount) of a bispecific antibody disclosed herein that is administered to a subject to inhibit EBOV infection will vary depending upon a number of factors associated with that subject, for example the overall health and/or weight of the subject. An effective amount can be determined by varying the dosage and measuring the resulting response, such as, for example, a reduction in EBOV titer. Effective amounts also can be determined through various in vitro, in vivo or in situ immunoassays.

An effective or therapeutically effective amount encompasses a fractional dose that contributes in combination with previous or subsequent administrations to attaining an effective response. For example, an effective amount of an agent can be administered in a single dose, or in several doses, for example daily, during a course of treatment lasting several days or weeks. However, the effective amount can depend on the subject being treated, the severity and type of the condition being treated, and the manner of administration. A unit dosage form of the bispecific antibody can be packaged in an amount, or in multiples of the effective amount, for example, in a vial (e.g., with a pierceable lid) or syringe having sterile components.

Epitope: An antigenic determinant. These are particular chemical groups or peptide sequences on a molecule that are antigenic (elicit a specific immune response). An antibody specifically binds a particular antigenic epitope on a polypeptide. In some examples, a disclosed bispecific antibody specifically binds to two different epitopes on EBOV GP (a first antigen binding portion of the bispecific antibody binds a first epitope of GP and a second antigen binding portion of the bispecific antibody binds a second epitope of GP).

Expression control sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcriptional terminators, a start codon (ATG) in front of a protein-encoding gene, splice signals for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Heterologous: Originating from a separate genetic source or species. For example, a promoter can be heterologous to an operably linked nucleic acid sequence.

Inhibiting a disease or condition: Reducing the full development of a disease or condition in a subject, for example, reducing the full development of EVD in a subject who has an EBOV infection (e.g., reducing viremia), and/or reducing EBOV infection in a subject or population of subjects at risk thereof. This includes neutralizing, antagonizing, prohibiting, preventing, restraining, slowing, disrupting, stopping, or reversing progression or severity of the disease or condition.

Inhibiting a disease or condition refers to a prophylactic intervention administered before the disease or condition has begun to develop (for example a treatment initiated in a subject at risk of an EBOV infection, but not infected by an EBOV) that reduces subsequent development of the disease or condition, and also to amelioration of one or more signs or symptoms of the disease or condition following development. The term “ameliorating,” with reference to inhibiting a disease or condition refers to any observable beneficial effect of the intervention intended to inhibit the disease or condition. The beneficial effect can be evidenced, for example, by a delayed onset of clinical symptoms of the disease or condition in a susceptible subject, a reduction in severity of some or all clinical symptoms of the disease or condition, a slower progression of the disease or condition, an improvement in the overall health or well-being of the subject, a reduction in infection, or by other parameters well known in the art that are specific to the particular disease or condition.

In some aspects, a bispecific antibody that specifically binds to EBOV GP inhibits infection of a human subject by an EBOV (such as Zaire ebolavirus), for example, by at least 25%, at least 50%, at least 60%, at least 70%, at least 80%, at least 90%, or at least 95%, compared to a control or compared to the absence of treatment.

Isolated: A biological component (such as a nucleic acid, peptide, protein or protein complex, for example an antibody or bispecific antibody) that has been substantially separated, produced apart from, or purified away from other biological components in the cell of the organism in which the component naturally occurs, that is, other chromosomal and extra-chromosomal DNA and RNA, and proteins. Thus, isolated nucleic acids, peptides and proteins include nucleic acids and proteins purified by standard purification methods. The term also embraces nucleic acids, peptides and proteins prepared by recombinant expression in a host cell, as well as, chemically synthesized nucleic acids. An isolated nucleic acid, peptide or protein, for example a bispecific antibody, can be at least 50%, at least 60%, at least 70%, at least 80%, at least 90%, at least 95%, at least 96%, at least 97%, at least 98%, or at least 99% pure.

Linker: A bi-functional molecule that can be used to link two molecules into one contiguous molecule, for example, to link an effector molecule to an antibody. Non-limiting examples of peptide linkers include glycine-serine linkers.

The terms “conjugating,” “joining,” “bonding,” or “linking” can refer to making two molecules into one contiguous molecule; for example, linking two polypeptides into one contiguous polypeptide, or covalently attaching an effector molecule or detectable marker radionuclide or other molecule to a polypeptide, such as an antibody or antibody fragment. The linkage can be either by chemical or recombinant means. “Chemical means” refers to a reaction between the antibody moiety and the effector molecule such that there is a covalent bond formed between the two molecules to form one molecule.

Neutralizing antibody: An antibody (or bispecific antibody) that reduces the infectious titer of an infectious agent by binding to a specific antigen on the infectious agent, such as a virus (e.g., EBOV). In some aspects, an antibody or bispecific antibody that is specific for EBOV GP neutralizes the infectious titer of EBOV. For example, an antibody or bispecific antibody that neutralizes EBOV may interfere with the virus by binding it directly and limiting entry into cells. Alternately, a neutralizing antibody may interfere with one or more post-attachment interactions of the pathogen with a receptor, for example, by interfering with viral entry using the receptor. In some aspects, an antibody or bispecific antibody that specifically binds to EBOV GP and neutralizes EBOV inhibits infection of cells, for example, by at least 50%, by at least 60%, by at least 70%, by at least 80% or by at least 90%, compared to a control antibody. In some aspects, an antibody, such as a bispecific antibody, that specifically binds to an EBOV GP can neutralize two or more (such as three, four, five, or more) species of Ebolavirus.

Nucleic acid (molecule or sequence): A deoxyribonucleotide or ribonucleotide polymer or combination thereof including without limitation, cDNA, mRNA, genomic DNA, and synthetic (such as chemically synthesized) DNA or RNA. The nucleic acid can be double stranded (ds) or single stranded (ss). Where single stranded, the nucleic acid can be the sense strand or the antisense strand. Nucleic acids can include natural nucleotides (such as A, T/U, C, and G), and can include analogs of natural nucleotides, such as labeled nucleotides.

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and the non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the disclosed bispecific antibodies.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, added preservatives (such as non-natural preservatives), and pH buffering agents and the like, for example sodium acetate or sorbitan monolaurate. In particular examples, the pharmaceutically acceptable carrier is sterile and suitable for parenteral administration to a subject for example, by injection. In some aspects, the active agent and pharmaceutically acceptable carrier are provided in a unit dosage form such as a pill or in a selected quantity in a vial. Unit dosage forms can include one dosage or multiple dosages (for example, in a vial from which metered dosages of the agents can selectively be dispensed).

Purified: The term purified does not require absolute purity; rather, it is intended as a relative term. Thus, for example, a purified peptide preparation (such as a purified bispecific antibody preparation) is one in which the peptide or protein (such as an antibody) is more enriched than the peptide or protein is in its natural environment within a cell. In one aspect, a preparation is purified such that the protein or peptide represents at least 50% of the total peptide or protein content of the preparation. Substantial purification denotes purification from other proteins or cellular components. A substantially purified protein is at least 60%, 70%, 80%, 90%, 95% or 98% pure. Thus, in one specific, non-limiting example, a substantially purified protein is 90% free of other proteins or cellular components.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In several aspects, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

Sequence identity: The identity between two or more nucleic acid sequences, or two or more amino acid sequences, is expressed in terms of the identity between the sequences. Sequence identity can be measured in terms of percentage identity; the higher the percentage, the more identical the sequences. Homologs and variants of a V_{L} or a V_{H} of an antibody that specifically binds a target antigen are typically characterized by possession of at least about 75%, for example at least about 80%, 85%, 90%, 91%, 92%, 93%, 94%, 95%, 96%, 97%, 98% or 99% sequence identity counted over the full-length alignment with the amino acid sequence of interest.

Methods of alignment of sequences for comparison are well known in the art. Various programs and alignment algorithms are described in: Smith and Waterman, Adv. Appl. Math. 2(4):482-489, 1981; Needleman and Wunsch, J. Mol. Biol. 48(3):443-453, 1970; Pearson and Lipman, Proc. Natl. Acad. Sci. U.S.A. 85(8):2444-2448, 1988; Higgins and Sharp, Gene, 73(1):237-244, 1988; Higgins and Sharp, Bioinformatics. 5(2):151-3, 1989; Corpet, Nucleic Acids Res. 16(22):10881-10890, 1988; Huang et al. Bioinformatics. 8(2):155-165, 1992; and Pearson, Methods Mol. Biol. 24:307-331, 1994. Altschul et al., J. Mol. Biol. 215(3):403-410, 1990, presents a detailed consideration of sequence alignment methods and homology calculations. The NCBI Basic Local Alignment Search Tool (BLAST) (Altschul et al., J. Mol. Biol. 215(3):403-410, 1990) is available from several sources, including the National Center for Biological Information and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site. Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100.

Specifically bind: When referring to an antibody or bispecific antibody, refers to a binding reaction which determines the presence of a target protein in the presence of a heterogeneous population of proteins and other biologics. Thus, under designated conditions, an antibody binds preferentially to a particular target protein, peptide or polysaccharide (such as an antigen present on the surface of a pathogen, for example EBOV GP) and does not bind in a significant amount to other proteins present in the sample or subject. Specific binding can be determined by methods known in the art. See Greenfield (Ed.), Antibodies: A Laboratory Manual, 2^{nd} ed. New York: Cold Spring Harbor Laboratory Press, 2014, for a description of immunoassay formats and conditions that can be used to determine specific immunoreactivity.

With reference to an antibody-antigen complex, specific binding of the antigen and antibody has a K_{D} of less than about 10⁻⁷ Molar, such as less than about 10⁻⁸ Molar, 10⁻⁹, or even less than about 10⁻¹⁰ Molar. K_{D} refers to the dissociation constant for a given interaction, such as a polypeptide-ligand interaction or an antibody-antigen interaction. For example, for the bimolecular interaction of an antibody or antigen binding fragment and an antigen it is the concentration of the individual components of the bimolecular interaction divided by the concentration of the complex.

An antibody (or antigen-binding fragment) that specifically binds to an epitope on EBOV GP is an antibody that binds substantially to EBOV GP, including cells or tissue expressing EBOV GP, substrate to which the EBOV GP is attached, or EBOV GP in a biological specimen. It is, of course, recognized that a certain degree of non-specific interaction may occur between an antibody or conjugate including an antibody (such as an antibody that specifically binds EBOV GP or conjugate including such antibody) and a non-target (such as a cell that does not express EBOV GP). Typically, specific binding results in a much stronger association between the antibody and protein or cells bearing the antigen than between the antibody and protein or cells lacking the antigen. Specific binding typically results in greater than 2-fold, such as greater than 5-fold, greater than 10-fold, or greater than 100-fold increase in amount of bound antibody (per unit time) to a protein including the epitope or cell or tissue expressing the target epitope as compared to a protein or cell or tissue lacking this epitope. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein.

Subject: Living multicellular vertebrate organisms, a category that includes human and non-human mammals. In some examples, the subject is a human. In other examples, the subject is a non-human primate. In some examples, the subject is a subject with an EBOV infection or at risk of an EBOV infection.

Transformed: A transformed cell is a cell into which a nucleic acid molecule has been introduced by molecular biology techniques. As used herein, the term transformed and the like (e.g., transformation, transfection, transduction, etc.) encompasses all techniques by which a nucleic acid molecule (such as an expression vector) might be introduced into such a cell, including transduction with viral vectors, transformation with plasmid vectors, and introduction of DNA by electroporation, lipofection, and particle gun acceleration.

Vector: An entity containing a nucleic acid molecule (such as a DNA or RNA molecule) bearing a promoter(s) that is operationally linked to the coding sequence of a protein of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses. In some aspects, a viral vector comprises a nucleic acid molecule encoding a disclosed bispecific antibody that specifically binds to EBOV GP.

### III. Bispecific Monoclonal Antibodies Specific for Ebola Virus Glycoprotein

Disclosed herein is the construction of a bispecific antibody that combines the antigen recognition sites of mAb114 and the mAb100-like mAbA09 (“A09” or “S1-4-A09”). mAbA09 is favorable because its interprotomer binding epitope is located at the base of GP, leaving enough space away from the epitope of mAb114, which is located at the apex of the GP. This enables the two Fabs of mAb114×A09 to bind EBOV GP simultaneously. The CrossMab format was used to make the bispecific IgG (mAb114×A09, also referred to as BiSp107). This format of BiSp107 was determined to produce the best yield and antigen binding characteristics. The functional equivalency between each arm of the bispecific antibody and the corresponding parent antibody was demonstrated. It is demonstrated herein that this design reduces the risk of glycoprotein mutagenesis and virus escape from the bispecific antibody. Using a NHP model, it is further demonstrated herein that BiSp107 prevents mortality when administered either before exposure or post-infection, and these effects require only a single dose.

mAb114 and mAbA09 heavy chain (HC) and light chain (LC) amino acid and nucleotide sequences are provided below and set forth herein as SEQ ID NOs: 1-8. In the amino acid sequences shown below, the residues of the leader peptide are in italics and the CDRs (determined using IMGT) are underlined. Bold residues indicate “hole” or “knob” substitutions in the CH3 domain for the CrossMab technology (see, e.g., US 2017/0129962).

mAb114 heavy chain (SEQ ID NO: 2)
MGWSCIILFLVATATGVHSEVQLVESGGGLIQPGGSLRLSCAASGFALRMYDMHWVRQTIDKRLEWVSAVGPSGDTY
YADSVKGRFAVSRENAKNSLSLQMNSLTAGDTAIYYCVRSDRGVAGLFDSWGQGILVTVSSASVAAPSVFIFPPSDE
QLKSGTASVVCLLNNFYPREAKVQWKVDNALQSGNSQESVTEQDSKDSTYSLSSTLTLSKADYEKHKVYACEVTHQG
LSSPVTKSFNRGECDKTHTCPPCPAPELLGGPSVFLFPPKPKDTLMISRTPEVTCVVVDVSHEDPEVKENWYVDGVE
VHNAKTKPREEQYNSTYRVVSVLTVLHQDWLNGKEYKCKVSNKALPAPIEKTISKAKGQPREPQVCTLPPSRDELTK
NQVSLSCAVKGFYPSDIAVEWESNGQPENNYKTTPPVLDSDGSFFLVSKLTVDKSRWQQGNVFSCSVMHEALHNHYT
QKSLSLSPGK

| | Leader | | | | | CL domain | CH2-CH3 |
|---|---|---|---|---|---|---|---|
| mAb114 HC | peptide | VH domain | H-CDR1 | H-CDR2 | H-CDR3 | (swapped) | domains* |
| Residues | 1-19 | 20-140 | 45-52 | 70-76 | 115-127 | 141-245 | 246-472 |

*Hole mutations shown in bold
mAb114 heavy chain CDR sequences
H-CDR1: GFALRMYD (SEQ ID NO: 12)
H-CDR2: VGPSGDT (SEQ ID NO: 13)
H-CDR3: VRSDRGVAGLFDS (SEQ ID NO: 14)
mAb114 light chain (SEQ ID NO: 4)
MGWSCIILFLVATATGVHSDIQMTQSPSSLSASVGDRITITCRASQAFDNYVAWYQQRPGKVPKLLISAASALHAGV
PSRFSGSGSGTHFTLTISSLQPEDVATYYCQNYNSAPLTFGGGTKVEIKSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVEPKSC

| | Leader | | | | | CH1 domain |
|---|---|---|---|---|---|---|
| mAb114 LC | peptide | VL domain | L-CDR1 | L-CDR2 | L-CDR3 | (swapped) |
| Residues | 1-19 | 20-130 | 46-51 | 69-71 | 108-116 | 131-231 |

mAb114 light chain CDR sequences
L-CDR1: QAFDNY (SEQ ID NO: 15)
L-CDR2: AAS (SEQ ID NO: 16)
L-CDR3: QNYNSAPLT (SEQ ID NO: 17)
A09 heavy chain (SEQ ID NO: 6)
MGWSCIILFLVATATGVHSQVQLQESGPGLVKPSETLSLTCVVSGGILSSFYWNWIRQPPGKGLEWIGNIYYSGSPN
YNASFQSRVAISVDTSKNQISLNLKSVTAADTAMYYCVRASRAYLWGSYRPTALDLWGQGSLVTVSSASTKGPSVFP
LAPSSKSTSGGTAALGCLVKDYFPEPVTVSWNSGALTSGVHTFPAVLQSSGLYSLSSVVTVPSSSLGTQTYICNVNH
KPSNTKVDKKVEPKSCDKTHTCPPCPAPELLGGPSVFLFPPKPKDTLMISRTPEVTCVVVDVSHEDPEVKENWYVDG
VEVHNAKTKPREEQYNSTYRVVSVLTVLHQDWLNGKEYKCKVSNKALPAPIEKTISKAKGQPREPQVYTLPPCRDEL
TKNQVSLWCLVKGFYPSDIAVEWESNGQPENNYKTTPPVLDSDGSFFLYSKLTVDKSRWQQGNVFSCSVMHEALHNH
YTQKSLSLSPGK

| | Leader | | | | | | CH2-CH3 |
|---|---|---|---|---|---|---|---|
| A09 HC | peptide | VH domain | H-CDR1 | H-CDR2 | H-CDR3 | CH1 domain | domains* |
| Residues | 1-19 | 20-144 | 45-52 | 70-76 | 115-133 | 145-247 | 248-474 |

*Knob mutations in bold
A09 heavy chain CDR sequences
H-CDR1: GGILSSFY (SEQ ID NO: 18)
H-CDR2: IYYSGSP (SEQ ID NO: 19)
H-CDR3: VRASRAYLWGSYRPTALDL (SEQ ID NO: 20)
A09 light chain (SEQ ID NO: 8)
MGWSCIILFLVATATGVHASYELTQPPSVSVSPGQTATITCSGDKLGDKYTSWFQQRPGQSPLLVIYQDNKRPSGLP
ARFSGSNSGNTATLTISGTQAMDEADYLCQVWDSGAVFGGGTKLTVLGQPKAAPSVTLFPPSSEELQANKATLVCLI
SDFYPGAVTVAWKADSSPVKAGVETTTPSKQSNNKYAASSYLSLTPEQWKSHRSYSCQVTHEGSTVEKTVAPTECS

| A09 LC | Leader peptide | VL domain | L-CDR1 | L-CDR2 | L-CDR3 | CL domain |
|---|---|---|---|---|---|---|
| Residues | 1-19 | 20-124 | 46-50 | 68-70 | 107-114 | 125-230 |

A09 light chain
L-CDR1: KLGDKY (SEQ ID NO: 21)
L-CDR2: QDN (SEQ ID NO: 22)
L-CDR3: QVWDSGAV (SEQ ID NO: 23)
mAb114 heavy chain nucleic acid sequence (SEQ ID NO: 1)
ATGGGCTGGTCCTGTATTATTCTGTTCCTGGTCGCAACTGCTACTGGGGTCCACTCCGAGGTGCAGCTGGTCGAAAG
CGGGGGGGGCCTGATCCAGCCAGGAGGCTCTCTGAGGCTGAGCTGCGCCGCCTCCGGCTTCGCCCTGAGGATGTACG
ACATGCACTGGGTGAGGCAGACCATCGATAAGCGCCTGGAGTGGGTGTCTGCCGTGGGACCTAGCGGCGACACATAC
TATGCCGATTCCGTGAAGGGCCGGTTTGCCGTGAGCCGGGAGAACGCCAAGAATAGCCTGTCCCTGCAGATGAACAG
CCTGACCGCCGGCGACACAGCCATCTACTATTGCGTGCGGAGCGATAGGGGCGTCGCAGGGCTGTTTGATTCTTGGG
GGCAGGGCATTCTGGTGACCGTGAGTTCAGCTAGCGTGGCAGCCCCTAGCGTGTTCATCTTTCCCCCTAGCGACGAA
CAGCTGAAGAGTGGAACTGCCTCCGTGGTGTGCCTGCTGAATAATTTCTATCCTAGGGAGGCCAAGGTCCAGTGGAA
AGTGGATAACGCTCTGCAGAGCGGGAATTCCCAGGAGTCTGTGACAGAACAGGACAGTAAGGATTCAACTTATAGCC
TGTCTAGTACACTGACTCTGTCCAAAGCAGACTACGAGAAGCACAAAGTCTATGCCTGCGAAGTGACTCATCAGGGG
CTGTCAAGCCCAGTGACCAAGTCTTTCAACCGCGGAGAATGTGACAAAACCCACACATGCCCACCATGTCCTGCACC
AGAGCTGCTGGGAGGACCAAGCGTGTTCCTGTTTCCTCCAAAGCCTAAAGATACTCTGATGATCTCCAGAACCCCCG
AAGTCACATGCGTGGTCGTGGACGTGTCTCACGAGGACCCCGAAGTCAAGTTTAACTGGTACGTGGACGGGGTCGAG
GTGCATAATGCCAAGACAAAACCCCGAGAGGAACAGTACAATAGCACCTATCGGGTCGTGTCCGTCCTGACAGTGCT
GCATCAGGATTGGCTGAACGGGAAGGAGTATAAGTGTAAAGTGAGTAATAAGGCACTGCCCGCCCCTATCGAGAAAA
CCATTAGCAAGGCTAAAGGACAGCCAAGAGAACCCCAGGTGTGCACACTGCCCCCTTCTAGGGACGAGCTGACTAAG
AACCAGGTCTCCCTGTCTTGTGCTGTGAAAGGCTTCTACCCTTCAGATATTGCAGTGGAGTGGGAAAGCAATGGGCA
GCCAGAAAACAATTATAAGACCACACCACCCGTGCTGGACTCCGATGGATCTTTCTTTCTGGTCTCTAAGCTGACCG
TGGATAAAAGTCGGTGGCAGCAGGGCAACGTCTTTAGTTGTTCAGTGATGCATGAGGCCCTGCACAATCATTACACA
CAGAAGAGCCTGTCCCTGTCTCCTGGCAAATGATGA
mAb114 light chain nucleic acid sequence (SEQ ID NO: 3)
ATGGGCTGGTCCTGTATTATCCTGTTCCTGGTCGCTACCGCAACCGGCGTCCACTCAGACATTCAGATGACTCAGTC
CCCTTCCTCCCTGTCTGCCAGCGTGGGCGACCGGATCACCATCACATGCAGAGCCAGCCAGGCCTTCGATAACTACG
TGGCCTGGTATCAGCAGAGGCCCGGCAAGGTGCCTAAGCTGCTGATCTCCGCCGCATCTGCCCTGCACGCAGGAGTG
CCATCCCGGTTCTCCGGATCTGGAAGCGGAACCCACTTTACCCTGACAATCAGCTCCCTGCAGCCAGAGGACGTGGC
AACATACTATTGTCAGAACTACAATTCCGCACCACTGACATTTGGGGGAGGCACTAAGGTCGAGATTAAGAGCAGCG
CATCAACCAAGGGCCCAAGCGTGTTCCCACTGGCTCCCTCAAGCAAGAGTACTTCAGGAGGAACCGCTGCACTGGGA
TGTCTGGTGAAAGATTATTTTCCCGAACCTGTGACCGTCAGCTGGAACTCCGGCGCCCTGACCAGCGGAGTGCACAC
TTTCCCTGCTGTCCTGCAGTCAAGCGGGCTGTATTCTCTGTCTTCAGTGGTCACCGTGCCAAGTTCAAGCCTGGGCA
CCCAGACATATATCTGCAACGTCAATCATAAGCCATCCAATACAAAAGTGGATAAGAAAGTCGAACCCAAATCTTGT
TGATGA
A09 heavy chain nucleic acid sequence (SEQ ID NO: 5)
ATGGGATGGTCATGTATCATCCTTTTTCTAGTAGCAACTGCAACCGGTGTACATTCCCAGGTCCAGCTGCAGGAAAG
CGGGCCAGGACTGGTCAAACCCTCCGAAACTCTGTCACTGACTTGCGTGGTCTCCGGCGGCATTCTGAGCTCCTTCT
ACTGGAACTGGATCAGGCAGCCACCTGGCAAGGGCCTGGAGTGGATCGGCAATATCTACTATTCTGGCAGCCCCAAC
TATAATGCCTCCTTTCAGTCTCGCGTGGCCATCAGCGTGGACACCTCCAAGAACCAGATCTCTCTGAATCTGAAGAG
CGTGACCGCCGCCGATACAGCCATGTACTATTGCGTGCGGGCATCCAGAGCATACCTGTGGGGCTCTTATCGGCCAA
CCGCACTGGATCTGTGGGGGCAGGGCTCACTGGTCACTGTCTCCTCAGCATCAACAAAGGGCCCAAGTGTGTTTCCC
CTGGCACCTTCTAGTAAATCAACTAGCGGAGGAACCGCAGCACTGGGATGTCTGGTGAAGGACTATTTCCCCGAGCC
TGTCACAGTGTCCTGGAACTCTGGCGCACTGACCAGCGGGGTCCACACATTTCCAGCCGTGCTGCAGTCAAGCGGCC
TGTACAGCCTGTCCTCTGTGGTCACTGTGCCCAGTTCAAGCCTGGGGACTCAGACCTATATCTGCAACGTGAATCAC
AAGCCAAGCAATACCAAAGTCGACAAGAAAGTGGAACCCAAGTCCTGTGATAAAACACATACTTGCCCTCCATGTCC
AGCACCAGAGCTGCTGGGAGGACCTAGCGTGTTCCTGTTTCCCCCTAAGCCAAAAGATACCCTGATGATTTCTCGGA
CACCCGAAGTCACTTGCGTGGTCGTGGACGTCAGTCACGAGGACCCCGAAGTCAAGTTCAACTGGTACGTGGACGGG
GTCGAGGTGCATAATGCTAAGACCAAACCCAGGGAGGAACAGTACAATTCAACTTATCGCGTCGTGAGCGTCCTGAC
CGTGCTGCACCAGGATTGGCTGAACGGAAAAGAGTACAAGTGTAAAGTGAGCAATAAGGCTCTGCCTGCACCAATCG
AGAAAACCATTAGCAAGGCCAAAGGCCAGCCAAGAGAACCCCAGGTGTACACACTGCCACCCTGCAGGGATGAGCTG
ACTAAGAACCAGGTCAGCCTGTGGTGTCTGGTGAAAGGGTTCTATCCTAGTGACATTGCTGTGGAGTGGGAATCAAA
TGGACAGCCAGAAAACAATTACAAGACCACACCTCCAGTGCTGGACAGTGATGGATCATTCTTTCTGTATTCCAAGC
TGACAGTGGACAAAAGCCGGTGGCAGCAGGGCAACGTCTTTTCCTGTTCTGTGATGCATGAGGCCCTGCACAATCAT
TACACTCAGAAGAGTCTGTCACTGAGCCCTGGCAAATGATGA
A09 light chain nucleic acid sequence (SEQ ID NO: 7)
ATGGGATGGTCATGTATCATCCTTTTTCTAGTAGCAACTGCAACCGGTGTCCATGCTTCCTATGAACTGACCCAGCC
CCCCAGCGTGAGCGTGAGCCCAGGCCAGACTGCCACCATTACTTGTAGCGGAGATAAACTGGGCGACAAGTACACAA
GCTGGTTCCAGCAGCGGCCCGGACAGTCCCCTCTGCTGGTCATCTACCAGGATAACAAGCGGCCATCTGGCCTGCCA
GCACGGTTCAGCGGCTCCAACTCTGGCAATACCGCCACACTGACCATCAGCGGAACCCAGGCAATGGACGAGGCAGA
TTACCTGTGTCAGGTCTGGGATAGCGGGGCTGTCTTCGGAGGGGGAACAAAACTGACTGTGCTGGGCGTCCTAGGTC
AGCCCAAGGCTGCCCCCTCGGTCACTCTGTTCCCGCCCTCCTCTGAGGAGCTTCAAGCCAACAAGGCCACACTGGTG
TGTCTCATAAGTGACTTCTACCCGGGAGCCGTGACAGTGGCCTGGAAGGCAGATAGCAGCCCCGTCAAGGCGGGAGT
GGAGACCACCACACCCTCCAAACAAAGCAACAACAAGTACGCGGCCAGCAGCTATCTGAGCCTGACGCCTGAGCAGT
GGAAGTCCCACAGAAGCTACAGCTGCCAGGTCACGCATGAAGGGAGCACCGTGGAGAAGACAGTGGCCCCTACAGAA
TGTTCATGA

Provided herein is a bispecific monoclonal antibody that specifically binds two distinct epitopes of EBOV GP. In some aspects, the bispecific monoclonal antibody includes (1) a first antigen binding portion comprising a heavy chain variable domain and a light chain variable domain, wherein the heavy chain variable domain comprises a heavy chain complementarity determining region (H-CDR)1, an H-CDR2 and an H-CDR3, and wherein the light chain variable domain comprises a light chain complementarity determining region (L-CDR)1, an L-CDR2 and an L-CDR3, wherein the H-CDR1, H-CDR2 and H-CDR3 are from the mAb114 heavy chain of SEQ ID NO: 2, and the L-CDR1, L-CDR2 and L-CDR3 are from the mAb114 light chain of SEQ ID NO: 4; and (2) a second antigen binding portion comprising a heavy chain variable domain and a light chain variable domain, wherein the heavy chain variable domain comprises an H-CDR1, an H-CDR2 and an H-CDR3, and wherein the light chain variable domain comprises an L-CDR1, an L-CDR2 and an L-CDR3, wherein the H-CDR1, H-CDR2 and H-CDR3 are from the A09 heavy chain of SEQ ID NO: 6, and the L-CDR1, L-CDR2 and L-CDR3 are from the A09 light chain of SEQ ID NO: 8.

The CDR positions can be determined using any appropriate numbering scheme, such as IMGT, Kabat, or Chothia. In some aspects, the CDRs are determined using IMGT.

In some aspects of the bispecific antibody, the amino acid sequences of the H-CDR1, H-CDR2 and H-CDR3 of the first antigen binding portion respectively comprise SEQ ID NO: 12, SEQ ID NO: 13 and SEQ ID NO: 14 (corresponding to residues 45-52, 70-76 and 115-127 of SEQ ID NO: 2, respectively); and/or the amino acid sequences of the L-CDR1, L-CDR2 and L-CDR3 of the first antigen binding portion respectively comprise SEQ ID NO: 15, SEQ ID NO: 16 and SEQ ID NO: 17 (corresponding to residues 46-51, 69-71 and 108-116 of SEQ ID NO: 4, respectively). In some examples, the amino acid sequence of the heavy chain variable domain of the first antigen binding portion is at least 80%, at least 85%, at least 90%, at least 95%, at least 96%, at least 97%, at least 98% or at least 99% identical to residues 20-140 of SEQ ID NO: 2; and/or the amino acid sequence of the light chain variable domain of the first antigen binding portion is at least 80%, at least 85%, at least 90%, at least 95%, at least 96%, at least 97%, at least 98% or at least 99% identical to residues 20-130 of SEQ ID NO: 4.

In some aspects, the amino acid sequences of the H-CDR1, H-CDR2 and H-CDR3 of the second antigen binding portion respectively comprise SEQ ID NO: 18, SEQ ID NO: 19 and SEQ ID NO: 20 (corresponding to residues 45-52. 70-76 and 115-133 of SEQ ID NO: 6, respectively); and/or the amino acid sequences of the L-CDR1, L-CDR2 and L-CDR3 of the second antigen binding portion respectively comprise SEQ ID NO: 21, SEQ ID NO: 22 and SEQ ID NO: 23 (corresponding to residues 46-50, 68-70 and 107-114 of SEQ ID NO: 8, respectively). In some examples, the amino acid sequence of the heavy chain variable domain of the second antigen binding portion is at least 80%, at least 85%, at least 90%, at least 95%, at least 96%, at least 97%, at least 98% or at least 99% identical to residues 20-144 of SEQ ID NO: 6; and/or the amino acid sequence of the light chain variable domain of the second antigen binding portion is at least 80%, at least 85%, at least 90%, at least 95%, at least 96%, at least 97%, at least 98% or at least 99% identical to residues 20-124 of SEQ ID NO: 8.

In some examples, the heavy chain variable domain of the first antigen binding portion comprises the H-CDR1, H-CDR2 and H-CDR3 of SEQ ID NO: 2 and the remaining residues of the heavy chain variable domain are at least 80%, at least 85%, at least 90%, at least 95%, at least 96%, at least 97%, at least 98% or at least 99% identical to residues 20-140 of SEQ ID NO: 2; and/or the light chain variable domain of the first antigen binding portion comprises the L-CDR1, L-CDR2 and L-CDR3 of SEQ ID NO: 4 and the remaining residues of the light chain variable domain are at least 80%, at least 85%, at least 90%, at least 95%, at least 96%, at least 97%, at least 98% or at least 99% identical to residues 20-130 of SEQ ID NO: 4.

In some examples, the heavy chain variable domain of the second antigen binding portion comprises the H-CDR1, H-CDR2 and H-CDR3 of SEQ ID NO: 6 and the remaining residues of the heavy chain variable domain are at least 80%, at least 85%, at least 90%, at least 95%, at least 96%, at least 97%, at least 98% or at least 99% identical to residues 20-144 of SEQ ID NO: 6; and/or the light chain variable domain of the second antigen binding portion comprises the L-CDR1, L-CDR2 and L-CDR3 of SEQ ID NO: 8 and the remaining residues of the light chain variable domain are at least 80%, at least 85%, at least 90%, at least 95%, at least 96%, at least 97%, at least 98% or at least 99% identical to residues 20-124 of SEQ ID NO: 8.

In specific examples, the amino acid sequence of the heavy chain variable domain of the first antigen binding portion comprises or consists of residues 20-140 of SEQ ID NO: 2; the amino acid sequence of the light chain variable domain of the first antigen binding portion comprises or consists of residues 20-130 of SEQ ID NO: 4; the amino acid sequence of the heavy chain variable domain of the second antigen binding portion comprises or consists of residues 20-144 of SEQ ID NO: 6; and/or the amino acid sequence of the light chain variable domain of the second antigen binding portion comprises or consists of residues 20-124 of SEQ ID NO: 8.

In some aspects, the first antigen binding portion, the second antigen binding portion, or both, are a Fab fragment, a Fab′ fragment, a single chain Fv protein (scFv), or a disulfide stabilized Fv protein (dsFv).

In some aspects, the first antigen binding portion of the bispecific monoclonal antibody includes a Fab comprising the heavy chain variable domain, the light chain variable domain, a heavy chain constant domain, and a light chain constant domain. In some examples, the heavy chain constant domain and the light chain constant domain are swapped. In specific examples, the amino acid sequence of the heavy chain constant domain is at least 80%, at least 85%, at least 90%, at least 95%, at least 96%, at least 97%, at least 98% or at least 99% identical to residues 131-231 of SEQ ID NO: 4; and/or the amino acid sequence of the light chain constant domain is at least 80%, at least 85%, at least 90%, at least 95%, at least 96%, at least 97%, at least 98% or at least 99% identical to residues 141-245 of SEQ ID NO: 2. In a particular non-limiting example, the amino acid sequence of the heavy chain constant domain comprises or consists of residues 131-231 of SEQ ID NO: 4; and/or the amino acid sequence of the light chain constant domain comprises or consists of residues 141-245 of SEQ ID NO: 2.

In some aspects, the second antigen binding portion of the bispecific monoclonal antibody includes a Fab comprising the heavy chain variable domain, the light chain variable domain, a heavy chain constant domain, and a light chain constant domain. In some examples, the amino acid sequence of the heavy chain constant domain is at least 80%, at least 85%, at least 90%, at least 95%, at least 96%, at least 97%, at least 98% or at least 99% identical to residues 145-247 of SEQ ID NO: 6; and/or the amino acid sequence of the light chain constant domain is at least 80%, at least 85%, at least 90%, at least 95%, at least 96%, at least 97%, at least 98% or at least 99% identical to residues 125-230 of SEQ ID NO: 8. In a particular non-limiting example, the amino acid sequence of the heavy chain constant domain comprises or consists of residues 145-147 of SEQ ID NO: 6; and/or the amino acid sequence of the light chain constant domain comprises or consists of residues 125-230 of SEQ ID NO: 8.

In some aspects, the first antigen binding portion further comprises a first CH2 domain and a first CH3 domain; and the second antigen binding portion further comprises a second CH2 domain and a second CH3 domain. In some examples, the amino acid sequence of the first CH2 domain and the first CH3 domain is at least 80%, at least 85%, at least 90%, at least 95%, at least 96%, at least 97%, at least 98% or at least 99% identical to residues 246-472 of SEQ ID NO: 2; and/or the amino acid sequence of the second CH2 domain and the second CH3 domain is at least 80%, at least 85%, at least 90%, at least 95%, at least 96%, at least 97%, at least 98% or at least 99% identical to residues 248-474 of SEQ ID NO: 6. In particular examples, the amino acid sequence of the first CH2 domain and the first CH3 domain comprises or consists of residues 246-472 of SEQ ID NO: 2; and/or the amino acid sequence of the second CH2 domain and the second CH3 domain comprises or consists of residues 248-474 of SEQ ID NO: 6.

In some aspects, the first antigen binding portion includes a heavy chain and a light chain, and the amino acid sequence of the heavy chain is at least 80%, at least 85%, at least 90%, at least 95%, at least 96%, at least 97%, at least 98% or at least 99% identical to residues 20-472 of SEQ ID NO: 2, and the amino acid sequence of the light chain is at least 80%, at least 85%, at least 90%, at least 95%, at least 96%, at least 97%, at least 98% or at least 99% identical to residues 20-131 of SEQ ID NO: 4; and/or the second antigen binding portion comprises a heavy chain and a light chain, and the amino acid sequence of the heavy chain is at least 80%, at least 85%, at least 90%, at least 95%, at least 96%, at least 97%, at least 98% or at least 99% identical to residues 20-474 of SEQ ID NO: 6, and the amino acid sequence of the light chain is at least 80%, at least 85%, at least 90%, at least 95%, at least 96%, at least 97%, at least 98% or at least 99% identical to residues 20-230 of SEQ ID NO: 8.

In specific examples, the first antigen binding portion comprises a heavy chain and a light chain, and the amino acid sequence of the heavy chain comprises or consists of residues 20-472 of SEQ ID NO: 2, and the amino acid sequence of the light chain comprises or consists of residues 20-131 of SEQ ID NO: 4; and/or the second antigen binding portion comprises a heavy chain and a light chain, and the amino acid sequence of the heavy chain comprises or consists of residues 20-474 of SEQ ID NO: 6, and the amino acid sequence of the light chain comprises or consists of residues 20-230 of SEQ ID NO: 8.

In some aspects, the Fc region of the bispecific antibody includes one or more amino acid substitutions to optimize in vivo half-life of the bispecific antibody. The serum half-life of IgG antibodies is regulated by the neonatal Fc receptor (FcRn). Thus, in several aspects, the bispecific antibody includes an amino acid substitution that increases binding to the FcRn. Several such substitutions are known to the person of ordinary skill in the art, such as substitutions at IgG constant regions T250Q and M428L (see, e.g., Hinton et al., J Immunol., 176:346-356, 2006); M428L and N434S (the “LS” mutation, see, e.g., Zalevsky, et al., Nature Biotechnology, 28:157-159, 2010); N434A (see, e.g., Petkova et al., Int. Immunol., 18:1759-1769, 2006); T307A, E380A, and N434A (see, e.g., Petkova et al., Int. Immunol., 18:1759-1769, 2006); and M252Y, S254T, and T256E (see, e.g., Dall' Acqua et al., J. Biol. Chem., 281:23514-23524, 2006).

Also provided herein are compositions that include a disclosed bispecific monoclonal antibody and a pharmaceutically acceptable carrier. Compositions are further described in section VI below.

Further provided are nucleic acid molecules that encode a bispecific monoclonal antibody disclosed herein, or encode a heavy chain, light chain, variable heavy domain or variable light domain disclosed herein. In some aspects, the nucleotide sequence of the heavy chain is at least 80%, at least 85%, at least 90%, at least 95%, at least 96%, at least 97%, at least 98% or at least 99% identical to SEQ ID NO: 1 or nucleotides 58-1422 of SEQ ID NO: 1; the nucleotide sequence of the light chain is at least 80%, at least 85%, at least 90%, at least 95%, at least 96%, at least 97%, at least 98% or at least 99% identical to SEQ ID NO: 3 or nucleotides 58-699 of SEQ ID NO: 3; the nucleotide sequence of the heavy chain is at least 80%, at least 85%, at least 90%, at least 95%, at least 96%, at least 97%, at least 98% or at least 99% identical to SEQ ID NO: 5 or nucleotides 58-1428 of SEQ ID NO: 5; or the nucleotide sequence of the light chain is at least 80%, at least 85%, at least 90%, at least 95%, at least 96%, at least 97%, at least 98% or at least 99% identical to SEQ ID NO: 7 or nucleotides 58-702 of SEQ ID NO: 7. In particular examples, the nucleotide sequence of the heavy chain comprises or consists of SEQ ID NO: 1 or nucleotides 58-1422 of SEQ ID NO: 1; the nucleotide sequence of the light chain comprises or consists of SEQ ID NO: 3 or nucleotides 58-699 of SEQ ID NO: 3; the nucleotide sequence of the heavy chain comprises or consists of SEQ ID NO: 5 or nucleotides 58-1428 of SEQ ID NO: 5; or the nucleotide sequence of the light chain comprises or consists of SEQ ID NO: 7 or nucleotides 58-702 of SEQ ID NO: 7. In some aspects, the nucleic acid molecule is operably linked to a promoter. Expression vectors that include a nucleic acid molecule disclosed herein, as well as host cells containing a disclosed nucleic acid or expression vector, are further provided. Nucleic acids, expression vectors and host cells are further described in section V below.

Also provided is a method of producing a bispecific monoclonal antibody that specifically binds Ebola virus glycoprotein. In some aspects, the method includes transfecting host cells with a first expression vector comprising the nucleotide sequence of SEQ ID NO: 1, a second expression vector comprising the nucleotide sequence of SEQ ID NO: 3, a third expression vector comprising the nucleotide sequence of SEQ ID NO: 5 and a fourth expression vector comprising the nucleotide sequence of SEQ ID NO: 7; and purifying the bispecific monoclonal antibody from the host cells and/or host cell culture supernatant. In some examples, the host cells are Expi293 cells.

Further provided are methods of preventing, inhibiting or treating an Ebola virus (EBOV) infection in a subject. In some aspects, the method includes administering to the subject a therapeutically effective amount of a bispecific monoclonal antibody or composition disclosed herein. In some examples, the subject has an EBOV infection. In particular examples in which the subject has an EBOV infection, the bispecific monoclonal antibody or composition is administered no more than 2, no more than 3, no more than 4, no more than 5, no more than 6, no more than 7, no more than 8, no more than 9, no more than 10, no more than 15 or no more than 20 days following EBOV infection. In other examples, the subject has been exposed to EBOV but has not been diagnosed as having an EBOV infection. In particular examples in which the subject has been exposed to EBOV but has not yet been diagnosed as having an EBOV infection, the bispecific monoclonal antibody or composition is administered no more than 2, no more than 3, no more than 4, no more than 5, no more than 6, no more than 7, no more than 8, no more than 9, no more than 10, no more than 15 or no more than 20 days following exposure to EBOV. In other examples, the subject has not yet been exposed to EBOV. In particular examples, in which the subject has not yet been exposed to EBOV, the bispecific monoclonal antibody or composition is administered about 16 weeks, about 14 weeks, about 12 weeks, about 10 weeks, about 8 weeks, about 6 weeks, about 4 weeks, about 2 weeks, about 1 one week, about 6 days, about 5 days, about 4 days, about 3 days, about 2 days and/or about 1 day prior to exposure to EBOV. Methods of use of the disclosed bispecific monoclonal antibodies are further described in section VII below.

### IV. Antigen-Binding Regions

The bispecific antibody (or an antibody included in an antigen binding portion of the bispecific antibody) can be of any isotype. The antibody can be, for example, an IgM or an IgG antibody, such as IgG₁ or an IgG₂. The class of an antibody that specifically binds, e.g., EBOV GP, can be switched with another. In one aspect, a nucleic acid molecule encoding V_{L} domain or V_{H} domain is isolated using methods well-known in the art, such that it does not include any nucleic acid sequences encoding the constant region of the light or heavy chain, respectively. The nucleic acid molecule encoding V_{L} or V_{H} is then operatively linked to a nucleic acid sequence encoding a C_{L} or C_{H} from a different class of immunoglobulin molecule. This can be achieved using a vector or nucleic acid molecule that includes a C_{L} or C_{H} chain, as known in the art. For example, an antibody that specifically binds EBOV GP that was originally IgM may be class switched to an IgG. Class switching can also be used to convert one IgG subclass to another, such as from IgG₁ to IgG₂.

An antigen binding portion included in the bispecific antibody can be a functional fragment (antigen binding fragment) of the antibodies described herein, such as an Fab fragment. The antibody fragments retain the ability to selectively bind with the antigen and can be included in a bispecific antibody. These fragments include:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;
(4) Fv, a genetically engineered fragment containing the V_{L} and V_{L} expressed as two chains; and
(5) Single chain antibody (such as scFv), defined as a genetically engineered molecule containing the V_{H} and the V_{L} linked by a suitable polypeptide linker as a genetically fused single chain molecule (see, e.g., Ahmad et al., Clin. Dev. Immunol., 2012, doi:10.1155/2012/980250; Marbry and Snavely, IDrugs, 13(8):543-549, 2010). The intramolecular orientation of the V_{H}-domain and the V_{L}-domain in a scFv, is not decisive for the provided antibodies (e.g., for the provided multispecific antibodies). Thus, scFvs with both possible arrangements (V_{H}-domain-linker domain-V_{L}-domain; V_{L}-domain-linker domain-V_{H}-domain) may be used.
(6) A dimer of a single chain antibody (scFV2), defined as a dimer of a scFv. This has also been termed a “miniantibody.”

Methods of making these fragments are known in the art (see for example, Greenfield (Ed.), Antibodies: A Laboratory Manual, 2^{nd} ed. New York: Cold Spring Harbor Laboratory Press, 2014).

For example, antigen binding fragments can be prepared by proteolytic hydrolysis of the antibody or by expression in a host cell (such as an E. coli cell) of DNA encoding the fragment. Antigen binding fragments can also be obtained by pepsin or papain digestion of whole antibodies by conventional methods. For example, antigen binding fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody. In some examples, the antibody heavy chain can include an engineered protease cleave site (such as an HRV3C protease cleavage site) in place of or in addition to the typical papain cleavage site to facilitate cleavage by proteases other than papain.

In some aspects, the antigen binding portion is a Fab, which contains a light chain (a VL domain and a constant domain) and a portion of a heavy chain (a VH domain and one constant domain).

### V. Nucleic Acid Molecules and Host Cells

Nucleic acid molecules encoding the amino acid sequences of the bispecific antibodies, or components thereof, that specifically bind EBOV GP are also provided herein. The nucleic acid molecules can encode a heavy chain or a fragment thereof (such as a heavy chain variable domain) and/or a light chain or a fragment thereof (such as a light chain variable domain). Exemplary nucleic acid sequences are set forth as SEQ ID NOs: 1, 3, 5 and 7. Recombinant nucleic acid molecules encoding bispecific antibodies, or a component thereof, can readily be produced by one of skill in the art, using the amino acid sequences provided herein, and the genetic code. In addition, one of skill can readily construct a variety of clones containing functionally equivalent nucleic acids, such as nucleic acids which differ in sequence but which encode the same protein sequence. Thus, nucleic acids encoding bispecific antibodies and their components, conjugates and fusion proteins are provided herein.

Nucleic acid sequences encoding bispecific antibodies, or a component thereof, that specifically bind EBOV GP can be prepared by any suitable method including, for example, cloning of appropriate sequences or by direct chemical synthesis by methods such as the phosphotricster method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. One of skill would recognize that while chemical synthesis of DNA is generally limited to sequences of about 100 bases, longer sequences may be obtained by the ligation of shorter sequences.

Exemplary nucleic acids encoding a bispecific antibody, or a component thereof, that specifically binds EBOV GP can be prepared by cloning techniques. Examples of appropriate cloning and sequencing techniques, and instructions sufficient to direct persons of skill through many cloning exercises are found in Sambrook et al., supra, Berger and Kimmel (eds.), supra, and Ausubel, supra. Product information from manufacturers of biological reagents and experimental equipment also provide useful information. Such manufacturers include the Sigma Chemical Company (Saint Louis, MO), R&D Systems (Minneapolis, MN), Pharmacia Amersham (Piscataway, NJ), CLONTECH Laboratories, Inc. (Palo Alto, CA), Chem Genes Corp., Aldrich Chemical Company (Milwaukee, WI), Glen Research, Inc., GIBCO BRL Life Technologies, Inc. (Gaithersburg, MD), Fluka Chemica-Biochemika Analytika (Fluka Chemie AG, Buchs, Switzerland), Invitrogen (San Diego, CA), and Applied Biosystems (Foster City, CA), as well as many other commercial sources known to one of skill.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

Once the nucleic acid molecules encoding a bispecific antibody, or a component thereof, that specifically binds EBOV GP are isolated and cloned, the protein can be expressed in a recombinantly engineered host cell such as a bacterial, plant, yeast, insect or mammalian cell (such as an Expi293 cell) using a suitable expression vector. One or more DNA sequences encoding the antibody, or a component thereof, can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

The expression of nucleic acid molecules encoding the bispecific antibodies (or portions thereof) described herein can be achieved by operably linking the DNA or cDNA to a promoter (which is either constitutive or inducible), followed by incorporation into an expression cassette. The promoter can be any promoter of interest, including a cytomegalovirus promoter. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signals for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned nucleic acid, it is desirable to construct expression cassettes which contain, for example, a strong promoter to direct transcription, a ribosome binding site for translational initiation (e.g., internal ribosomal binding sequences), and a transcription/translation terminator. For E. coli, this can include a promoter such as the T7, trp, lac, or lambda promoters, a ribosome binding site, and a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by any suitable method such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

Modifications can be made to nucleic acid molecules encoding a bispecific antibody described herein without diminishing its biological activity. Some modifications can be made to facilitate the cloning, expression, or incorporation of the antibody into a fusion protein. Such modifications include, for example, termination codons, sequences to create conveniently located restriction sites, and sequences to add a methionine at the amino terminus to provide an initiation site, or additional amino acids (such as poly His) to aid in purification steps.

Once expressed, the bispecific antibodies can be purified according to standard procedures in the art, including ammonium sulfate precipitation, affinity columns, column chromatography, and the like (see, generally, Simpson et al. (Eds.), Basic methods in Protein Purification and Analysis: A Laboratory Manual, New York: Cold Spring Harbor Laboratory Press, 2009). The bispecific antibodies need not be 100% pure. Once purified, partially or to homogeneity as desired, if to be used therapeutically, the antibodies should be substantially free of endotoxin.

Methods for expression of polypeptides, antibodies, fusion proteins, etc., and/or refolding to an appropriate active form, from mammalian cells, and bacteria such as E. coli have been described and are applicable to the antibodies disclosed herein. See, e.g., Greenfield (Ed.), Antibodies: A Laboratory Manual, 2^{nd} ed. New York: Cold Spring Harbor Laboratory Press, 2014, Simpson et al. (Eds.), Basic methods in Protein Purification and Analysis: A Laboratory Manual, New York: Cold Spring Harbor Laboratory Press, 2009, and Ward et al., Nature 341(6242):544-546, 1989.

### VI. Compositions

Compositions are provided that include the EBOV GP bispecific monoclonal antibody (or portions thereof, such as a heavy chain and/or a light chain) disclosed herein in a carrier. The compositions are useful, for example, for the prevention, inhibition or treatment of an EBOV infection. The compositions can be prepared in unit dosage forms for administration to a subject. The amount and timing of administration are at the discretion of the administering physician to achieve the desired purposes. The EBOV GP bispecific antibody can be formulated for systemic or local administration. In one example, the bispecific antibody is formulated for parenteral administration, such as intravenous administration, for example by injection or infusion.

In some aspects, the bispecific antibody in the composition is at least 70% (such as at least 75%, at least 80%, at least 85%, at least 90%, at least 95%, at least 96%, at least 97%, at least 98% or at least 99%) pure. In some aspects, the composition contains less than 10% (such as less than 5%, less than 4%, less than 3%, less than 2%, less than 1%, less than 0.5%, or even less) of macromolecular contaminants, such as other mammalian (e.g., human) proteins.

The compositions for administration can include a solution of the EBOV GP bispecific antibody dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of bispecific antibody in these formulations can vary widely, and will be selected primarily based on fluid volumes, viscosities, body weight and the like in accordance with the particular mode of administration selected and the subject's needs.

A typical composition for intravenous administration includes about 0.01 to about 30 mg/kg of bispecific antibody per subject per day. Actual methods for preparing administrable compositions are known and are described in more detail in such publications as Remington: The Science and Practice of Pharmacy, 22^{nd} ed., London, UK: Pharmaceutical Press, 2013. In some aspects, the composition can be a liquid formulation including the bispecific antibody in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml.

Bispecific antibodies can be provided in lyophilized form and rehydrated with sterile water before administration, although they are also provided in sterile solutions of known concentration. The bispecific antibody solution can then be added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of Rituximab in 1997. Antibodies (including bispecific antibodies) can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by daily or weekly maintenance doses of 2 mg/kg infused over a 30-minute period if the previous dose was well tolerated.

Controlled-release parenteral formulations can be made as implants, oily injections, or as particulate systems. For a broad overview of protein delivery systems see, Banga, Therapeutic Peptides and Proteins: Formulation, Processing, and Delivery Systems, Lancaster, PA: Technomic Publishing Company, Inc., 1995. Particulate systems include microspheres, microparticles, microcapsules, nanocapsules, nanospheres, and nanoparticles. Microcapsules contain the active protein agent, such as a cytotoxin or a drug, as a central core. In microspheres, the active protein agent is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, Colloidal Drug Delivery Systems, J. Kreuter (Ed.), New York, NY: Marcel Dekker, Inc., pp. 219-342, 1994; and Tice and Tabibi, Treatise on Controlled Drug Delivery: Fundamentals, Optimization, Applications, A. Kydonieus (Ed.), New York, NY: Marcel Dekker, Inc., pp. 315-339, 1992.

Polymers can be used for ion-controlled release of the antibody compositions disclosed herein. Various degradable and nondegradable polymeric matrices for use in controlled drug delivery are known in the art (Langer, Acc. Chem. Res. 26(10):537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res., 9(3):425-434, 1992; and Pec et al., J. Parent. Sci. Tech., 44(2):58-65, 1990). Alternatively, hydroxyapatite has been used as a microcarrier for controlled release of proteins (Ijntema et al., Int. J. Pharm. 112(3):215-224, 1994). In yet another aspect, liposomes are used for controlled release as well as drug targeting of the lipid-capsulated drug (Betageri et al., Liposome Drug Delivery Systems, Lancaster, PA: Technomic Publishing Co., Inc., 1993). Numerous additional systems for controlled delivery of active protein agents are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

### VII. Method of Use

Methods are disclosed herein for the prevention, inhibition or treatment of an EBOV infection (such as Zaire ebolavirus infection). The methods include administering to a subject a therapeutically effective amount (that is, an amount effective to prevent, inhibit or treat an EBOV infection in a subject) of a disclosed bispecific antibody to a subject infected with EBOV or at risk of EBOV infection (such as Zaire ebolavirus infection). The methods can be used pre-exposure or post-exposure. In some instances, the subject has been exposed to EBOV, but has not yet been diagnosed as having an EBOV infection and/or has not developed any signs or symptoms of having an EBOV infection (post-exposure prophylaxis). In other instances, the subject has been diagnosed with an EBOV infection (post-exposure treatment). In yet other cases, the subject has not been exposed to EBOV, but is at risk (such as high risk) of a future exposure (pre-exposure prophylaxis).

In some aspects of the disclosed methods, the subject has an EBOV infection. In some examples, the bispecific monoclonal antibody or composition is administered no more than 2, no more than 3, no more than 4, no more than 5, no more than 6, no more than 7, no more than 8, no more than 9, no more than 10, no more than 15 or no more than 20 days following EBOV infection.

In other aspects of the disclosed methods, the subject has been exposed to EBOV but has not been diagnosed as having an EBOV infection. In some examples, the bispecific monoclonal antibody or composition is administered no more than 2, no more than 3, no more than 4, no more than 5, no more than 6, no more than 7, no more than 8, no more than 9, no more than 10, no more than 15 or no more than 20 days following exposure to EBOV.

In yet other aspects of the disclosed methods, the subject has not yet been exposed to EBOV. In some examples, the bispecific monoclonal antibody or composition is administered about 16 weeks, about 14 weeks, about 12 weeks, about 10 weeks, about 8 weeks, about 6 weeks, about 4 weeks, about 2 weeks, about 1 one week, about 6 days, about 5 days, about 4 days, about 3 days, about 2 days and/or about 1 day prior to exposure to EBOV.

The disclosed antibodies can be administered to the subject alone, or in combination with other antibodies that target EBOV antigens to inhibit EBOV infection in the subject. In some aspects, a disclosed bispecific antibody is administered to the subject in combination with ZMapp, MIL77E and/or REGN-EB3 to inhibit an EBOV infection (such as Zaire ebolavirus infection) in the subject.

The EBOV infection does not need to be completely eliminated or inhibited for the method to be effective. For example, the method can inhibit infection by a desired amount, for example by at least 10%, at least 20%, at least 50%, at least 60%, at least 70%, at least 80%, at least 90%, at least 95%, at least 98%, or even at least 100% (elimination or prevention of detectable EBOV infection) as compared to EBOV infection in the absence of the treatment. In some examples, the method can reduce transient viremia by at least 10%, at least 20%, at least 50%, at least 60%, at least 70%, at least 80%, at least 90%, at least 95%, at least 98%, or even at least 100% (elimination or prevention of transient viremia) as compared to EBOV infection in the absence of the treatment.

In some aspects, administration of an effective amount of a disclosed bispecific antibody or composition thereof inhibits the establishment of EBOV infection and/or subsequent EVD progression in a subject, which can encompass any statistically significant reduction in EBOV activity or symptoms of EBOV infection in the subject.

Antibodies (such as bispecific antibodies) are typically administered by intravenous infusion; however, other routes of administration are possible and contemplated herein. Doses of the bispecific antibody can vary, but generally range between about 0.5 mg/kg to about 50 mg/kg, such as a dose of about 1 mg/kg, about 5 mg/kg, about 10 mg/kg, about 20 mg/kg, about 30 mg/kg, about 40 mg/kg, or about 50 mg/kg. In some aspects, the dose of the bispecific antibody can be from about 0.5 mg/kg to about 5 mg/kg, such as a dose of about 1 mg/kg, about 2 mg/kg, about 3 mg/kg, about 4 mg/kg or about 5 mg/kg. The bispecific antibody is administered according to a dosing schedule determined by a medical practitioner. In some examples, the bispecific antibody is administered twice per day, daily, weekly, every two weeks, every three weeks or every four weeks.

Single or multiple administrations of a composition including a disclosed EBOV GP bispecific antibody or composition thereof, can be administered depending on the dosage and frequency as required and tolerated by the patient. The dosage can be administered once, but may be applied periodically until either a desired result is achieved or until side effects warrant discontinuation of therapy. Generally, the dose is sufficient to inhibit EBOV infection without producing unacceptable toxicity to the patient.

Data obtained from cell culture assays and animal studies can be used to formulate a range of dosage for use in humans. The dosage normally lies within a range of circulating concentrations that include the ED₅₀, with little or minimal toxicity. The dosage can vary within this range depending upon the dosage form employed and the route of administration utilized. The effective dose can be determined from cell culture assays and animal studies.

The EBOV GP bispecific antibody, or a composition thereof, can be administered to subjects in various ways, including local and systemic administration, such as, e.g., by injection subcutaneously, intravenously, intra-arterially, intraperitoneally, intramuscularly, intradermally, or intrathecally. In an aspect, the bispecific antibody, or a composition thereof, is administered by a single subcutaneous, intravenous, intra-arterial, intraperitoneal, intramuscular, intradermal or intrathecal injection once a day, such as for a total of 1, 2, 3, 4 or 5 days. A further method of administration is by osmotic pump (e.g., an Alzet pump) or mini-pump (e.g., an Alzet mini-osmotic pump), which allows for controlled, continuous and/or slow-release delivery of the bispecific antibody over a pre-determined period. The osmotic pump or mini-pump can be implanted subcutaneously, or near a target site.

### EXAMPLES

The following examples are provided to illustrate particular features of certain aspects of the disclosure, but the scope of the claims should not be limited to those features exemplified.

### Example 1: Materials and Methods

This example describes the materials and experimental procedures for the studies described in Examples 2-8.

### Production of Fab Fragments

Fab fragments were produced using the Fab Preparation Kit (Pierce) or with HRV3C proteolytic cleavage (McLellan et al., Nature 336-343, 2011). HRV3C protease cleavage was used to produce Fab from antibodies with a heavy-chain harboring an HRV3C site at the hinge. mAb was bound to protein A sepharose and washed three times with PBS. About 0.5 column volume of PBS was added to the resin and 10 units of HRV3C (Novagen) per mg of IgG was added. The column was capped and gently mixed for 4 hours at room temperature. The unbound fraction was collected by gravity over a His-pur resin (Thermo) column to remove the HRV3C. The columns were washed with three volumes of PBS to collect remaining Fab. Initial and wash fractions were combined, concentrated with Amicon Ultra 10,000 MWCO filter unit and dialyzed against PBS.

### Plasmids

The pCAGGS-GCN4-Avi vector was created by synthesizing DNA (Integrated DNA Technologies) encoding for a GCN4 site followed by an Avitag peptide (underlined) and His tags (MKQIEDKIEEILSKIYHI ENEIARIKKLIGEVASSSGLNDIFEAQKIEWHEAHHHH HHG; SEQ ID NO: 9) and cloned into the pCAGGS expression vector using EcoRI and SmaI (New England Biolabs). EBOV N terminal probe-GCN4-His-SA (Ebola virus variant Mayinga GP, Δ309-505, Δ657-676) was codon optimized for human cell expression, synthesized (Genscript) and cloned into a pCAGGS expression vector (Misasi et al., Science 351:1343-1346, 2016). Soluble EBOV N terminal probe was amplified by polymerase chain reaction (PCR) using a primer pair: ATGGTACCTAAATGGGCGTTACAGGA (SEQ ID NO: 10) and CGACGCGTTCCAATACCTGCCGGT (SEQ ID NO: 11). The PCR products were subsequently cloned into pCAGGS-GCN4-Avi using KpnI and MluI (New England Biolabs). The EBOV Avitag proteins were expressed in HEK293T cells and then purified (Misasi et al., Science 351:1343-1346, 2016; Corti et al., Science 351:1339-1342, 2016). mAb100 and mAb114 heavy chains were previously reported (Corti et al., Science 351:1339-1342, 2016) and cloned into VRC8400 (CMV/R expression vector)-based IgG1 vector or a vector containing an HRV3C protease site at the hinge of IgG1 heavy chain for creation of Fabs (McLellan et al., Nature 336-343, 2011). mAb100 light chain was cloned into a CMV/R-based lambda chain expression vector and mAb114 light chain was cloned into a CMV/R-based kappa chain expression vector. The NPC1-domain C monomer vector expresses residues 371-621 of NPC1 (domain C) in between two GCN4 trimerization domains. Specifically, the vector sequence encodes N′-CD5 signal peptide-GCN4 domain-linker-inverted GCN4 domain-domain C-GCN4 domain-HA/His tags-C′.

### NPC1-Domain C Expression and Purification

NPC1-domain C plasmid was transfected into 293T cells using Lipofectamine 2000 (Life Technologies). The media was changed after 24 hours to 293SFMII supplemented with 2 mM CaCl₂, 1× non-essential amino acids and 1× penicillin/streptomycin (Life Technologies). Media was harvested daily for 4 more days afterwards. Protein supernatants were clarified by centrifugation at 3,273×g for 10 minutes and filtered. The protein was purified using Ni2+-NTA resin. NPC1-domain C protein used for kinetics and competition experiments was further purified by size-exclusion chromatography using a Superdex 200 column (GE Healthcare).

### Parental Antibodies, Antibody Production and Purification

KZ52 and 13C6 were purchased from IBT Bioservices. VRC01 was acquired via NIH AIDS Reagent Program. Antibodies mAb114, mAbA09, mAb100 were made by transfecting heavy and light chain plasmids at a 1:1 ratio using 293Fectin into 293Freestyle or Expi293 cells. The culture medium was supplemented with 10% volume of AbBooster (ABI Scientific) after 24 hours of incubation. Supernatants were harvested 5 days later, clarified by centrifugation at 3273×g for 30 minutes and passed through a 0.22 μm filter. Antibody was purified using Protein A Sepharose (GE Healthcare), eluted with IgG Elution buffer (Pierce), neutralized with 1/10th volume of 1 M Tris (pH9.0) and concentrated using Amicon Ultra centrifugal filter units (Millipore). Concentrated antibody was dialyzed against PBS using Slide-A-Lyzer 10,000 MWCO (Pierce). Unless otherwise indicated, isotype control antibody was an anti-HIV-1 gp120 IgG1 monoclonal that was produced as described above.

### Bispecific Antibody Production and Purification

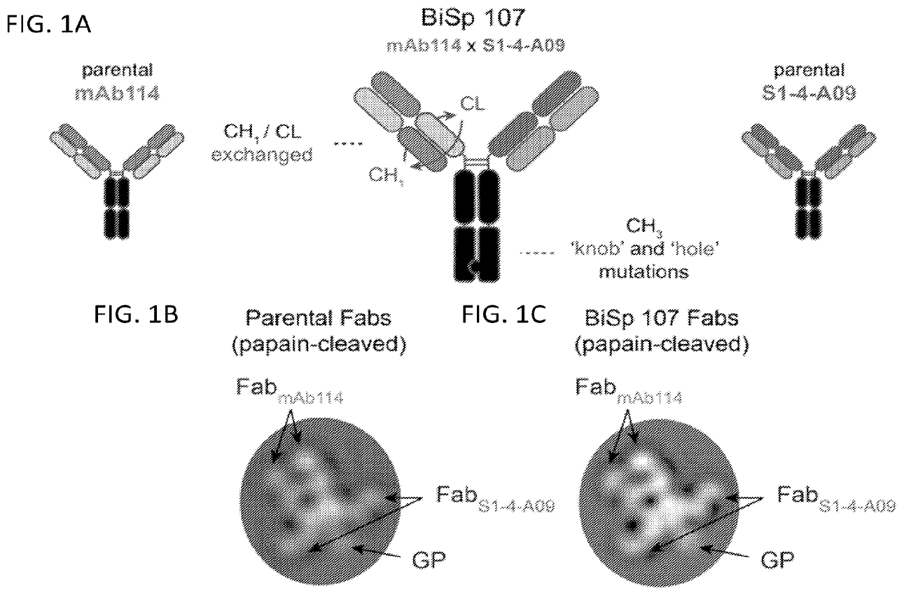

DNA plasmids encoding the four chains of the BiSp107 CrossMab bispecific antibody (mAb114 heavy chain with hole mutations and CH1-CL swap, mAb114 light chain with CH1-CL swap, A09 heavy chain with knob mutations and A09 light chain; FIG. 1A) were prepared either commercially by Genscript or by maxiprep according to the manufacturer's instructions (Origene). A titration of different ratios of the four plasmids encoding the fragments to create the full bispecific antibody were transfected into Expi293 cells (Invitrogen) according to the manufacturer's instructions (A14526, Invitrogen). The cell culture supernatant was harvested 5 days later, clarified by centrifugation at 3690 rpm for 30 minutes and filtered through a 0.22 μm sterile filter. The antibody was isolated using Protein A Sepharose (17-0974-04, GE Healthcare), eluted with IgG elution buffer (21004, Thermofisher), neutralized with 1/10th volume Tris pH 8.5 and concentrated with Amicon Ultra centrifugal filter units (Millipore Sigma). Buffer exchange was performed in the centrifugal filter units with PBS pH7.4. The antibody isolated from each individual culture was prepared in duplicate with NuPAGE LDS sample buffer (Invitrogen) with one sample being reduced with DTT for 5 minutes at 70° C. and the second remaining non-reduced. A total of 2 μg of protein was used in each sample. The samples were loaded on a NuPAGE 4-12% Bis-Tris gel (Invitrogen) and run at 120 V for 120 minutes. Gels were stained with InstantBlue protein stain (Expedeon).

### Production of GPTHL

Cleaved GP (GPTHL) was produced using thermolysin as previously described (Misasi et al., Science 351: 1343-1346, 2016).

### Protease Protection Assays

For the Cathepsin L (Cat L) protease protection assay, EBOV N terminus probe-GCN4-His-SA (560 nM) was incubated with 3.2 μM of mAb in reaction buffer (100 mM sodium acetate, 50 mM NaCl, 5 mM DTT, pH5.5). Prior to the addition of enzyme, 1/10th of the solution was removed for time 0 min. Cat L (Athen's Research) was added at a concentration of 0.2 units/mg of GP and incubated at 37° C. Samples were removed at 10, 20 and 30 minutes and transferred into tubes containing stop solution whose final concentration yielded 1×SDS loading buffer, 50 mM DTT, 250 mM HEPES, pH7.4 and 1× protease inhibitor cocktail (Roche). Samples were immediately boiled and subsequently analyzed using immunoblot for GP1 as described above. For the thermolysin protease protection assay, EBOV N terminus probe-GCN4-His-SA (560 nM) pre-incubated with 3.2 μM of mAb was digested using 0.02 mg/ml thermolysin (Sigma) in NT Buffer (10 mM Tris-Cl, pH 7.5, 135 mM NaCl) at 37° C. Prior to the addition of enzyme, 1/10th of the solution was removed for time 0 min. Six samples were removed at 3 minute intervals into tubes containing Stop solution. Samples were immediately boiled and subsequently analyzed using immunoblot for GP1 as described above.

### Biolayer Interferometry Binding Kinetics Measurement

BiSp107 binding kinetics to EBOV N-terminus probe and full probe were measured using a FortéBio Octet HTX instrument. The AR2G biosensors (amine-reactive 2nd generation, fortéBio) were activated by a mixture of 1-ethyl-3-(3-dimethylaminopropyl) carbodiimide hydrochloride (EDC) and N-hydroxysuccinimade (NHS) (diluted in water at 3.75 and 1.15 mg/ml, respectively) for 300 seconds. EBOV probes (10 μg/ml in 10 mM Acetate, pH 4.5) were captured on activated AR2G biosensors through amine-coupling reaction for 600 seconds. Typical capture levels after quenching with 1 M ethanolamine (pH 8.0) for 300 seconds were between 1.2 and 1.6 nm, and variability within the same protein did not exceed 0.15 nm. Biosensors were then equilibrated for 420 seconds in PBS-BSA prior to binding assessment of BiSp107. Association of the mAb114 arm of BiSp107 to the N-terminus probe (75 to 2.3 nM in PBS-BSA) was measured for 300 seconds and dissociation was measured for 300-3,600 seconds in PBS-BSA. FabA09 was used for side by side comparison. To measure the association of the A09 arm of BiSp107 to full probe, the probe was prebound with miAb114 (666.6 nM) and the association to full probe (150 to 2.3 nM in PBS-BSA) was measured for 300 seconds. Dissociation was measured for 300-3,600 seconds in PBS-BSA. Fab114 was used for side by side comparison. Correction of non-specific baseline drift was carried out by subtracting the measurements recorded for a sensor loaded with HIV-1 gp120 incubated with the parental Fabs or the BiSp107. Data analysis and curve fitting were carried out using Octet analysis software, version 8.0-9.0. Experimental data were fitted using a 1:1 binding model for all experiments. Global analyses of the complete data sets assuming binding was reversible (full dissociation) were carried out using nonlinear least-squares fitting allowing a single set of binding parameters to be obtained simultaneously for all concentrations used in each experiment.

### Bispecificity Assay

BiSp107 bispecificity was analyzed on a FortéBio Octet RED384 instrument. AR2G biosensors (18-5092, FortéBio) were activated by a mixture of 1-ethyl-3-(3-dimethylaminopropyl) carbodiimide hydrochloride (EDC) and N-hydroxysuccinimide (NHS) (diluted in water at 3.75 and 1.15 mg/ml, respectively) for 300 seconds. The N-terminal GP dimer (10 μg/ml in 10 mM acetate, pH 4.5) was captured on activated AR2G biosensors through an amine-coupling reaction for 600 seconds or until a 2.5 nm shift threshold was met, then quenched with ethanolamine for 300 seconds. The biosensors were then equilibrated in blocking buffer (PBS+1% BSA+0.01% Tween+0.02% sodium azide) for 450 seconds before probing into either the bispecific or parent antibodies (25 and 6.25 μg/ml) for 75 seconds to assess the binding to N-terminal GP. Following the first association with the antibodies, the biosensors were then probed into full length EBOV glycoprotein (50 μg/ml in blocking buffer) for 600 seconds to evaluate the ability of the unengaged Fab to bind to the full-length GP. Finally, the biosensors were equilibrated again in blocking buffer for 600 seconds to observe any dissociation. All of the traces were aligned to the baseline following the quenching step. Data were exported from Octet analysis software and analyzed on GraphPad Prism.

### Neutralization Assay

Parental mAbs and BiSp107 were evaluated for their capacity to neutralize pseudotyped lentivirus particles expressing at their surface EBOV GP (Mayinga variant U31033) as described previously (Corti et al., Science 351: 1339-1342, 2016). Briefly, luciferase activity was detected in HEK293T cell lysate upon lentivector infection in the presence or absence of mAb serially diluted with concentrations ranging from 6.67×10⁻⁰⁸ to 6.67×10⁻¹¹ mol/L. The relative luminescence unit (RLU) was measured using luciferase assay system Bright-Glo™ (Promega) and an EnVision plate reader (PerkinElmer). The percentage infectivity was calculated using the last mAb dilution point of 6.67×10⁻¹¹ mol/L as reference for each mAb analyzed.

### Enzyme-Linked Immunosorbent Assay

Methods for the GP IgG ELISA have been described previously (Sullivan et al., Nature 424: 681-684, 2003). Briefly, polyvinyl chloride ELISA plates (Dynatech, Vienna, VA, or Nunc, Rochester, NY) were coated with Ebola full length probe, washed, and incubated with antibodies (mab114×A09, mab114, mabA09 and VRC01). Bound antibody was detected using goat anti-human IgG (H+L; Chemicon/Millipore, Billerica, MA) conjugated to horseradish peroxidase and Sigma Fast o-phenylenediamine dihydrochloride (Sigma, St. Louis, MO). The HIV anti-gp120 antibody VRC01 is used as a negative control.

### Animal Study and Safety

Rhesus macaques 3-5 years old and weighing between 3-5 kg were obtained from Covance for the immunization and challenge studies. All experiments involving the use of ZEBOV in animals were performed in a BSL-4 laboratory. Animals were housed individually, and given enrichment regularly. Subjects were anesthetized with ketamine prior to blood sampling or antibody administration. Animals were randomly assigned to treatment groups based on sequential selection from a population inventory. Investigators were blinded to investigational antibodies but not treatment status. The treatment group contained three rhesus macaques. Challenge studies included a single untreated animal (control); the use of historical controls (n>50) allows for one untreated control to be used in each challenge experiment. Animals were transferred one week prior to challenge to the BSL-4 facility for exposure to a lethal (1000 PFU) i.m. EBOV Kikwit variant challenge.

### Macaque Vaccination and Infection

All animals were challenged by the intramuscular route with 1000 PFU of EBOV Kikwit. Five days post challenge, BiSp107 was administered by intravenous injection in peripheral veins using <20 gauge butterfly needles over a period of >15 minutes in a single bolus via syringe pump with the dosage of 100 mg/kg, 50 mg/kg or 25 mg/kg. Three animals were in each dose group and one macaque was not treated with bispecific antibody as a negative control animal. For the prophylaxis NHP group, BiSp107 was administered at a dose of 50 mg/kg at 3, 2 and 1 day before the EBOV Kikwit challenge of 1000 PFU.

### Statistics

Differences in survival outcome were compared by log rank test using GraphPad Prism 5.0 software. Sample sizes of three animals per Bio-Safety Level-4 (BSL4) EBOV challenge group provide 80% power to detect a difference in survival rates assuming 100% survival (3/3 treated survive) vs. 0% survival in negative controls at the 95% confidence level (1-tailed Fisher exact test).

### Example 2: Design of BiSp107 Antibody

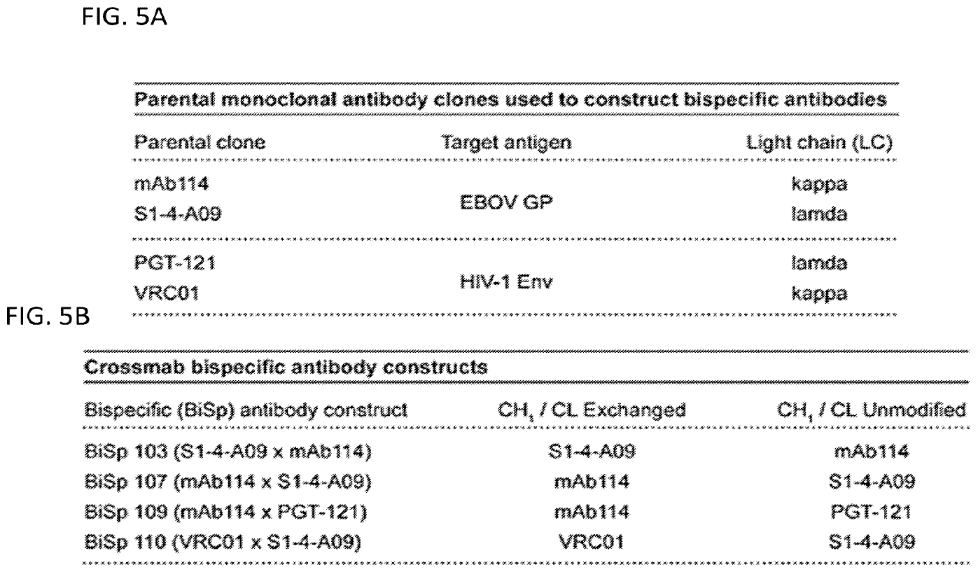

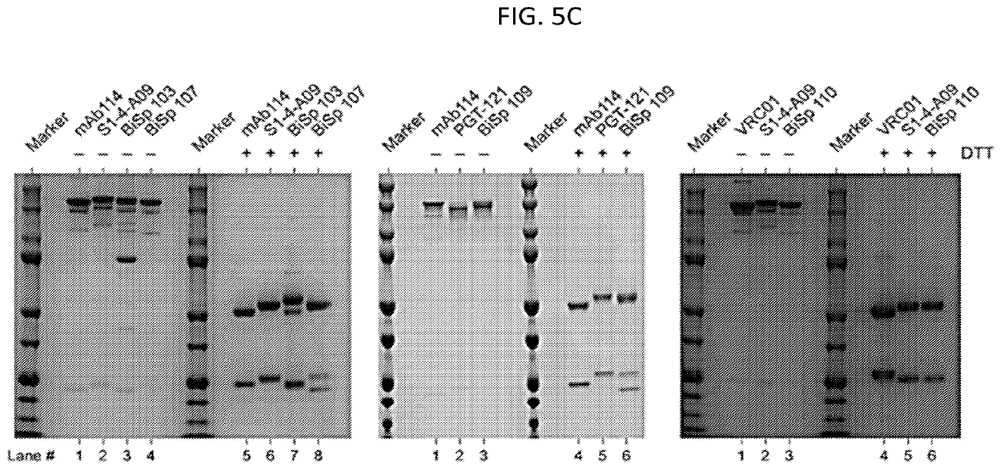

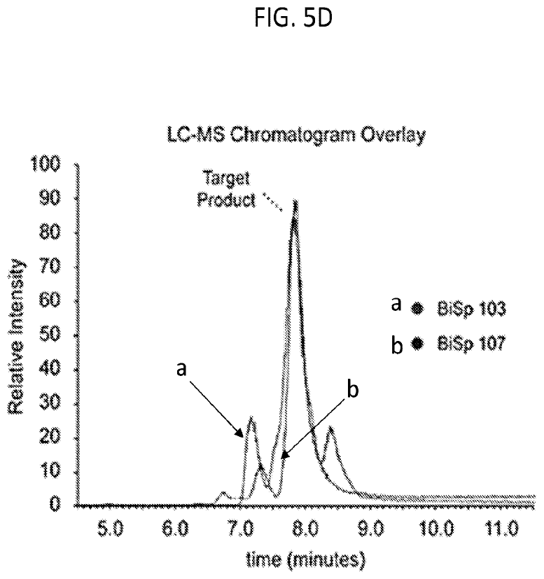

BiSp107 (mAb114×A09) was designed using CrossMab CH1-CL format as described previously (Schaefer et al., Proc Natl Acad Sci USA 108:11187-11192, 2011, Asokan et al., J Virol 89:12501-12512, 2015). To determine the optimal format and configuration of the components, several bispecific formats were tested, including the BiTE format and alternate configurations of the mAb114 component within the CrossMab format. The particular arrangement of BiSp107 in the CrossMab format was determined to produce the most efficient yield and best antigen binding. The CH1-CL swap of BiSp107 was performed on the mAb114 arm along with associated hole mutations in the CH3 region. The A09 arm was unswapped but contained knob mutations in the CH3 region (FIG. 1A). A second bispecific antibody mAbA09×mAb114 (BiSp103) was constructed to identify which bispecific antibody would have better purity for manufacture. BiSp107 was chosen to move forward into in vivo NHP platform verification because BiSp103 had more side products compared to BiSp107. Control bispecific antibodies were made in the same orientation by replacing one of the functional arms with HIV-specific antibodies (FIGS. 5A-5B). Plasmid ratios for transfection were optimized in order to obtain fully assembled bispecific antibodies with a minimal amount of partially assembled and mis-assembled products. SDS-PAGE and mass spectrometry were used to confirm that a bispecific antibody was obtained (FIGS. 5C-5D). With the ratio of HC1(mAb114): LC1(mAb114):HC2(A09):LC2(A09)=1:2:3.5:7, the least amount of heavy chain dimers and half antibodies were generated.

Negative-stain electron microscopy and single particle analysis were performed to compare the binding epitopes of Fab114 and FabA09 from respective parental mAbs (FIG. 1B) and Fab114 and FabA09 from BiSp107 (FIG. 1C). Fab114 from BiSp107 binds GP with a near-vertical angle of approach within the chalice of GP and with three Fabs per trimer simultaneously. FabA09 from Bisp107 binds to the base of the GP trimer and made contact with GP1 and GP2 of one protomer. These images demonstrated that the Fabs angles of approach are similar between BiSp107 and parental mAbs.

### Example 3: Binding Characteristics of BiSP107

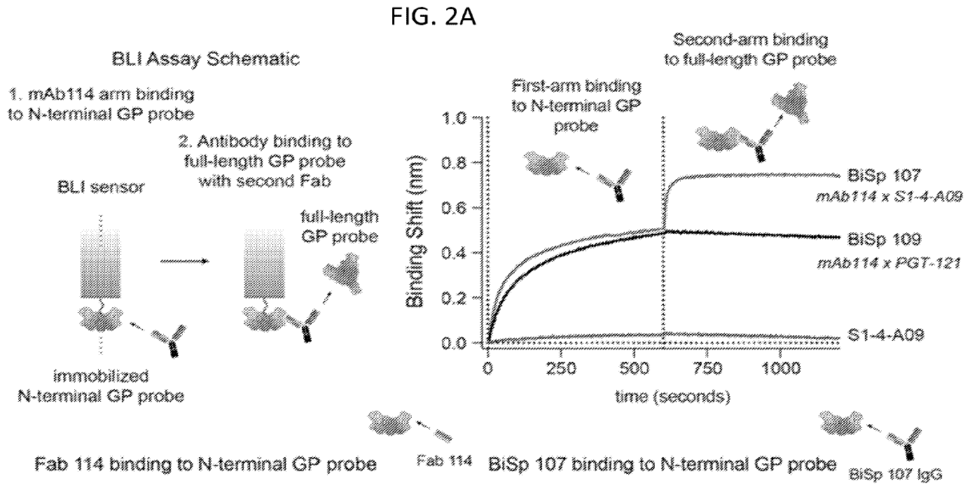

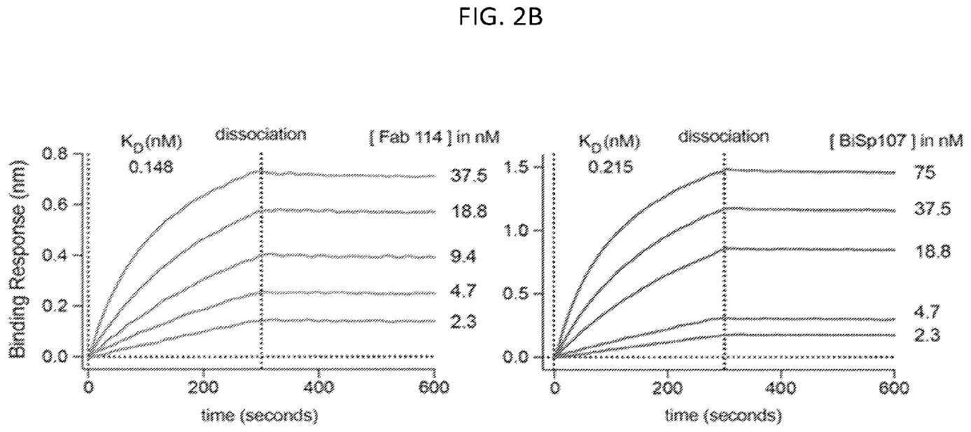

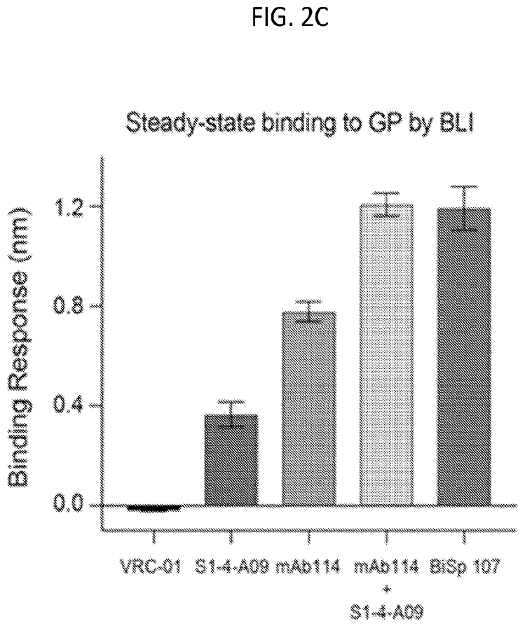

BiSp107 was assessed using biolayer interferometry in a sequential sandwich format between a 364 aa long N-terminal probe containing only the mAb114 epitope and a 676 aa long full-length GP probe containing both mAb114 and mAbA09 epitopes. The bispecific antibody was engaged by the surface-bound N-terminal probe and subsequently was also able bind the full-length probe. In comparison, parental mAb114 and control bispecific mAb114×PGT121 (BiSp109) only bound the N-terminal probe in the first step and not the full-length probe in the second step. Parental mAbA09 did not bind to N-terminal probe because it does not contain the mAbA09 epitope. Together, these data confirm that the bispecific antibody contained both the cap-binding and base-binding activities of mAb114 and A09 respectively, within a single molecule (FIG. 2A).

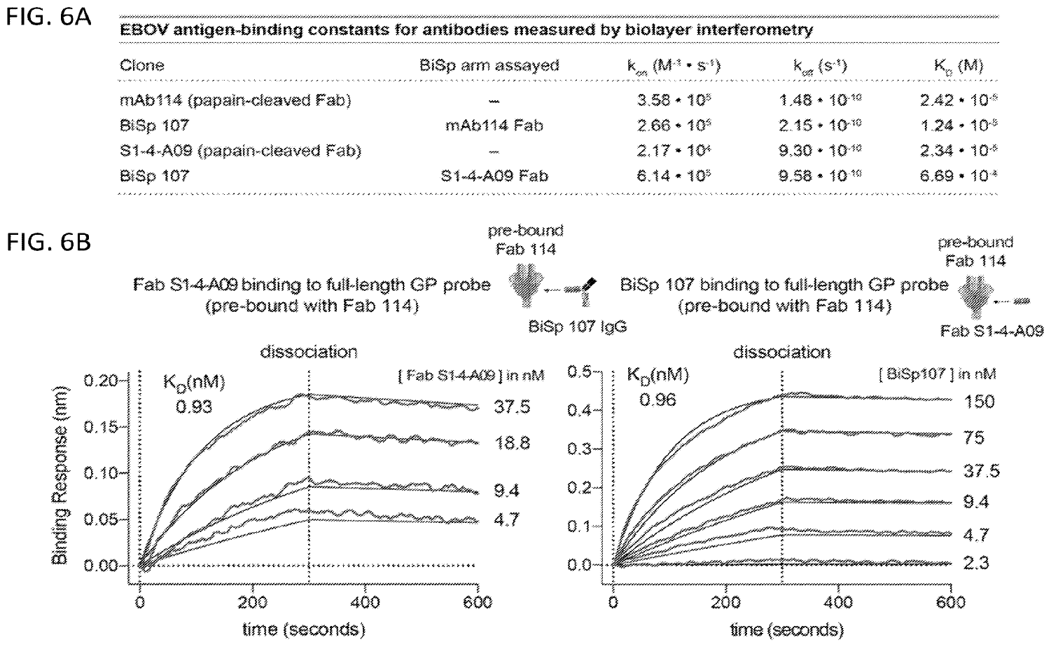

Because BiSp107 was designed with a swapped Fab114 arm, a study was conducted to determine whether this format would impact the binding characteristics. The affinity, on-rate and off-rate of the bispecific antibody in comparison to its parental arms were evaluated (FIG. 2B and FIGS. 6A-6B). BiSp107 antibody and Fab114 bound with similar kinetics to the N-terminal probe. Binding of the mAbA09 arm was assessed on full-length GP in which the glycan cap was blocked with excess Fab114 and only the base region was available for binding. The on-rate of FabA09 was 3.4-fold quicker than BiSp107, though the off-rates were comparable. Overall, each arm of BiSp107 had similar (within 3-fold difference) binding kinetics to their parental Fabs.

Next, it was evaluated whether BiSp107 could saturate all the binding sites on the EBOV full-length GP (FIG. 2C). Using biolayer interferometry with full probe coated on the sensor, antibodies were bound until saturation and the final binding responses were compared. In line with the predicted number of binding sites, two-fold more binding by mAb114 than A09 was observed. The physical mixture of mAb114 and A09 bound to the same level as the sum of mAb114 and A09 individually. The binding response from the bispecific antibody was similar to the two parental antibodies mixture, indicating that BiSp107 was able to fully occupy all the binding sites on full-length GP.

### Example 4: Functional Characterization of BiSp107

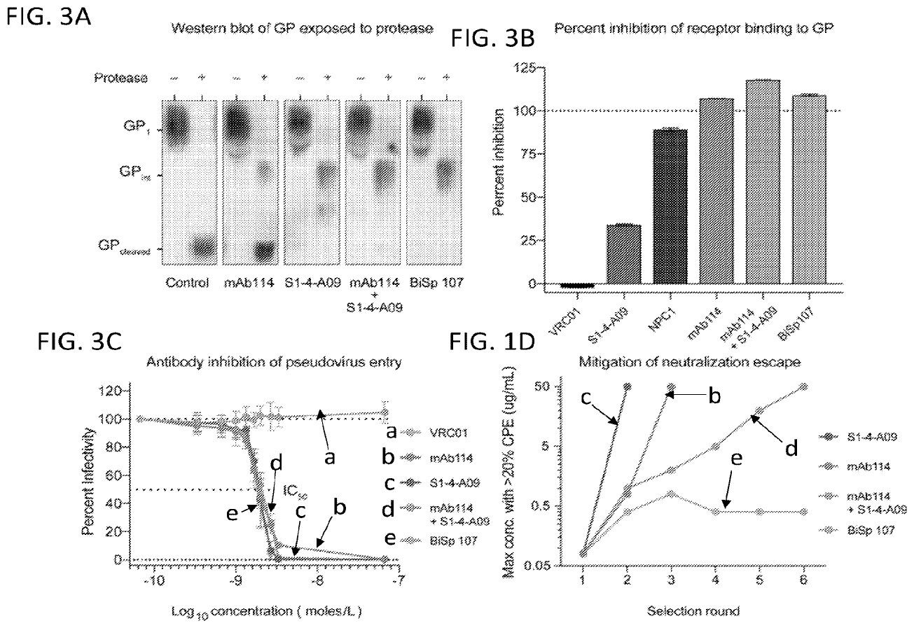

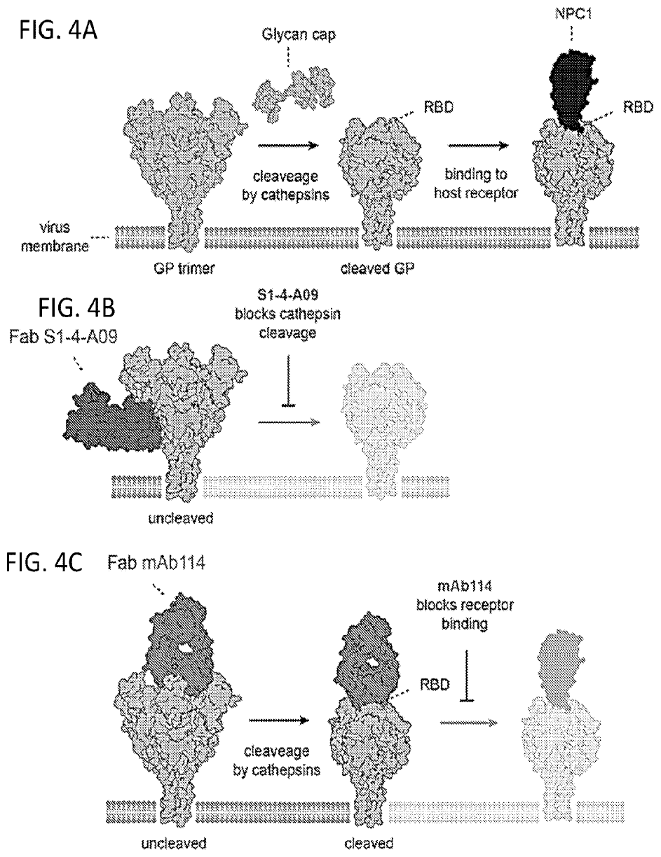

As described above, the bispecific antibody retains similar binding properties as its parental mAb. This example evaluates whether the bispecific antibody also retains the functional characteristic of parental mAb. It was next evaluated whether the bispecific IgG could inhibit the cleavage of the EBOV GP by thermolysin, which mimics the cathepsin-mediated cleavage of GP in the proteasomes and lysosomes (FIG. 3A). It has been previously shown that mAb100 can decrease the rate of GP1 cleavage resulting in the significantly delayed appearance of intermediate form GP1_{Intermediate} (Misasi et al., Science 351: 1343-1346, 2016). The present study demonstrates that bispecific IgG prevented the digestion of GP1_{cleaved} to GP_{THL} from the cathepsin and delayed the advent of intermediate form GP1_{Intermediate}, similar to parental mAbA09.

The receptor binding domain (RBD) of EBOV GP is exposed after cathepsin cleavage of GP1. It was previously reported that mAb114 was able to bind GP_{THL}, which blocked the NPC1 access to the receptor binding domain (RBD) pocket. Hence, the ability of BiSp107 to similarly block the GP_{THL} binding to its receptor NPC1 was evaluated in a bio-layer interferometry assay (FIG. 3B). KZ52 and 13C6 do not block RBD binding as described previously (Misasi et al., Science 351: 1343-1346, 2016). BiSp107 blocked the NPC1 binding to GP_{THL} completely, similarly to either mAb114 alone or the mixture of mAb114 and mAbA09.

As final confirmation that BiSp107 can prevent viral infection, its neutralization potency in comparison to the parental mAbs was investigated (FIG. 3C). Lentiviral particles pseudotyped with EBOV GP were used to assess virus entry inhibition in vitro (Corti et al., Science 351: 1339-1342, 2016). As demonstrated previously, mAb114 and mAbA09 fully neutralized lentivector particles at 6.67e−8 moles/L. The addition of both mAbs as a cocktail (mAb114+mAbA09) did not improve the neutralization capacity beyond the use of a single mAb. BiSp107 fully neutralized EBOV GP pseudotyped particles in the similar fashion and concentration range as the parental mAbs.

During the course of infection, escape mutants might be generated, thus it was investigated whether BiSp107 decreases the in vitro generation of escape mutant and mitigating resistance development. The capacity of BiSp107 to prevent the appearance of EBOV-induced cytopathic effect (CPE) through multiple rounds of passaging in the presence of increasing concentrations of antibodies was evaluated.
mAbA09: acquired full resistance of 50 μg/ml within 1st round;
mAb114: acquired partial resistance at 1st round, full resistance at 2nd round;
cocktail: acquisition of resistance was gradual, with less than an order of magnitude in the 1st several rounds;
BiSp107: modest resistance, remained sensitive to 50 μg/ml concentration throughout 6 rounds, it can block resistance acquisition, and the result is consistent with the neutralization result.

### Example 5: Synergistic Mitigation of Escape Acquisition by BiSp107

During viral infections, viruses can be selected that decrease or limit the effectiveness of therapeutic treatments (e.g., escape). One approach to decrease escape risk is to combine therapeutics with distinct mechanisms of action together in order to limit the risk that a single mutation will knockout the function of each component (e.g., triple anti-retroviral therapy in HIV). For monoclonal antibodies, efforts have focused on reducing escape risk by combining 2 or more monoclonal antibodies together in a single treatment. To determine the capacity of an antibody or a combination of antibodies to prevent escape, antibodies were incubated in the presence of replication competent vesicular stomatitis virus (rcVSV) bearing the Ebolavirus glycoprotein. Serial passaging of rcVSV Ebola was then performed in the presence of increasing dilutions of S1-4-A09, mAb114, the combination of S1-4-A09 and mAb114, or BiSp107 and virus replication was measured by the presence of >20% cytopathic effect (CPE) (FIG. 3D). The results showed that within two selection passaging rounds, rcVSV Ebola fully escaped neutralization at the highest concentration (50 micrograms/mL) by mAb114 and S1-4-A09 (FIG. 3D). In contrast, the combination of S1-4-A09 with an equal amount of mAb114 delayed the rate of complete escape (>20% CPE at 50 micrograms/mL) until round 6. Strikingly, virus growth in the presence of BiSp107 only occurred at concentrations of BiSp107 (˜0.4 μg/mL) that would not be expected to fully neutralize virus growth (around the IC50), suggesting that BiSp107 prevents escape (FIG. 3D). Since the combination of mAb114 and S1-4-A09 did not similarly prevent escape, this data shows that BiSp107 is synergistically neutralizing and mitigating neutralization escape in a manner would not be predicted and represents a novel finding.

### Example 6: BiSp107 Shows Similar PK Properties to mAb114 in Human FcR Mice

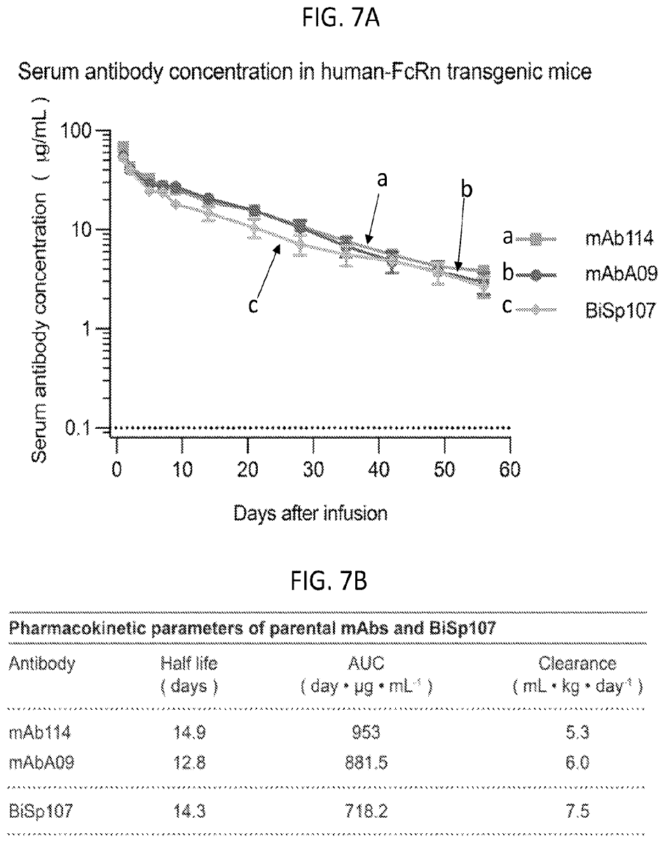

Since pharmacokinetics (PK) properties of antibodies can influence antibody treatment effectiveness, an initial evaluation of the PK properties of BiSp107 in comparison to mAb114 and miAbA09 was performed using a mouse engineered to express the human-FcRn. All three antibodies had similar PK properties with half-lives between 12.8-14.9 days (FIGS. 7A-7B), which suggests that BiSp107 will act similarly to mAb114 in humans.

### Example 7: Post-Infection Treatment by BiSp107

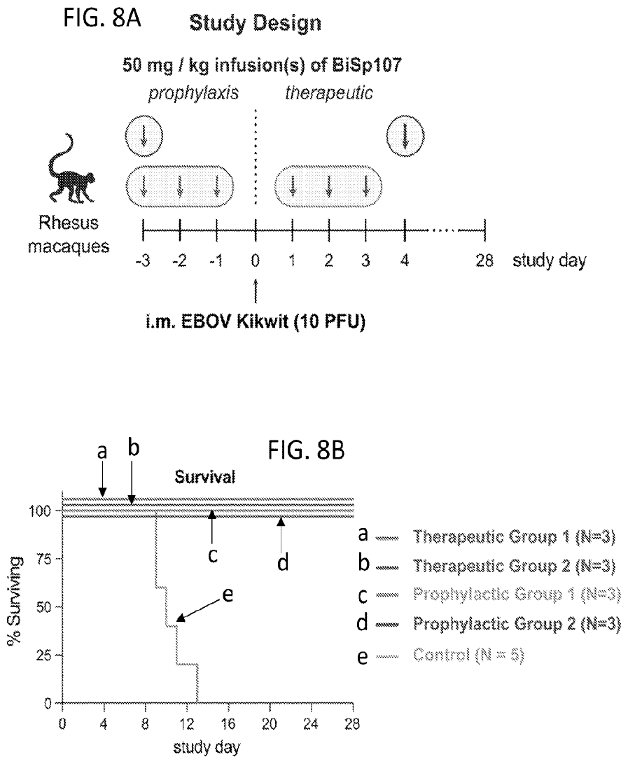

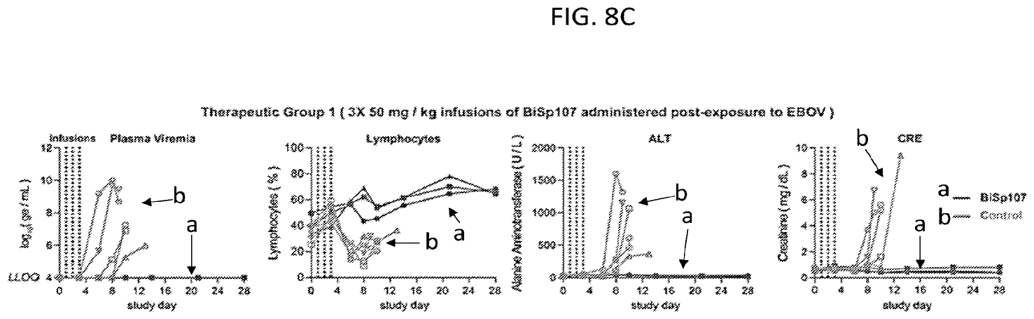

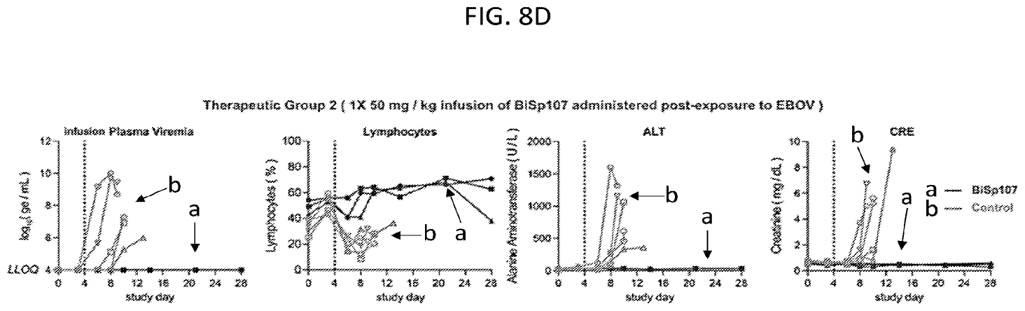

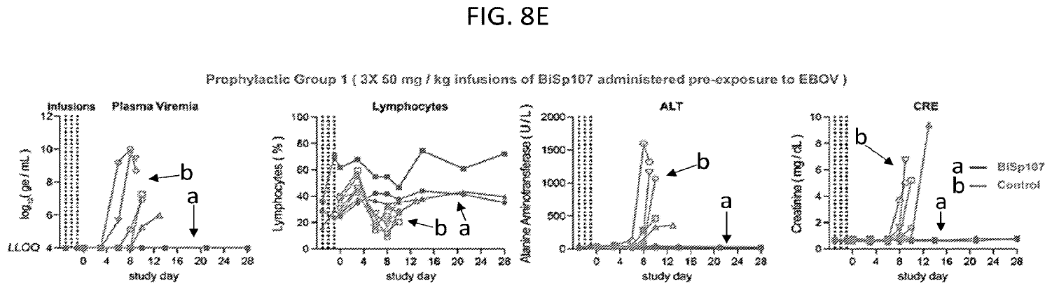

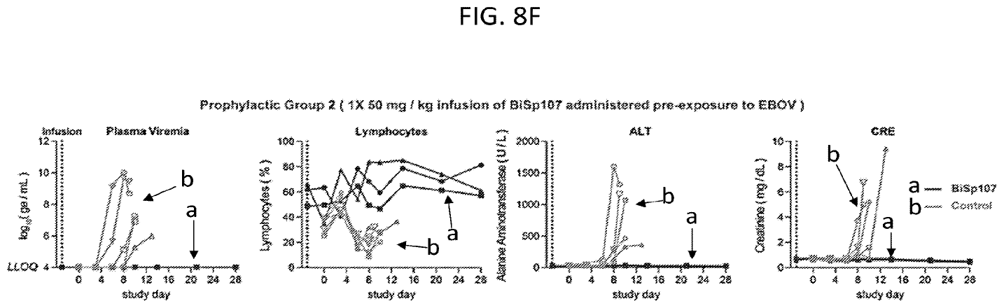

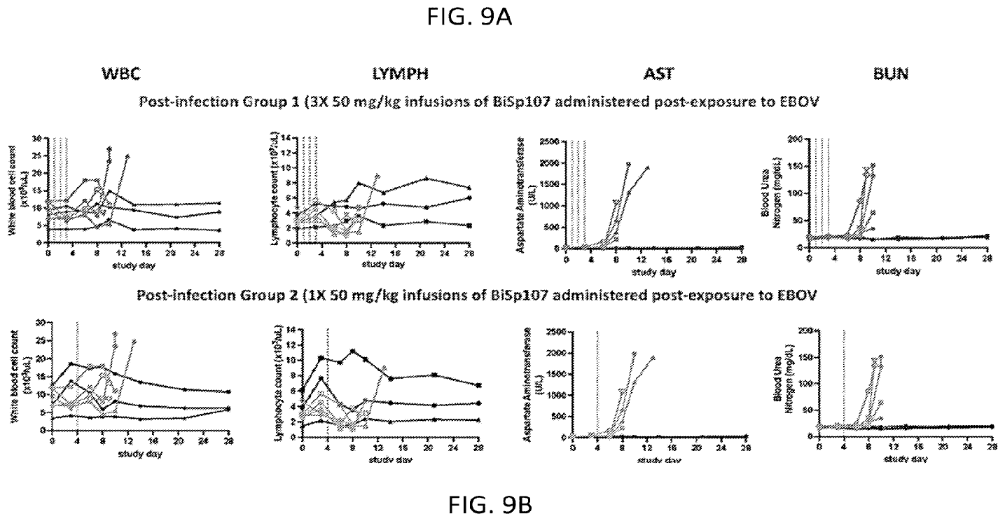

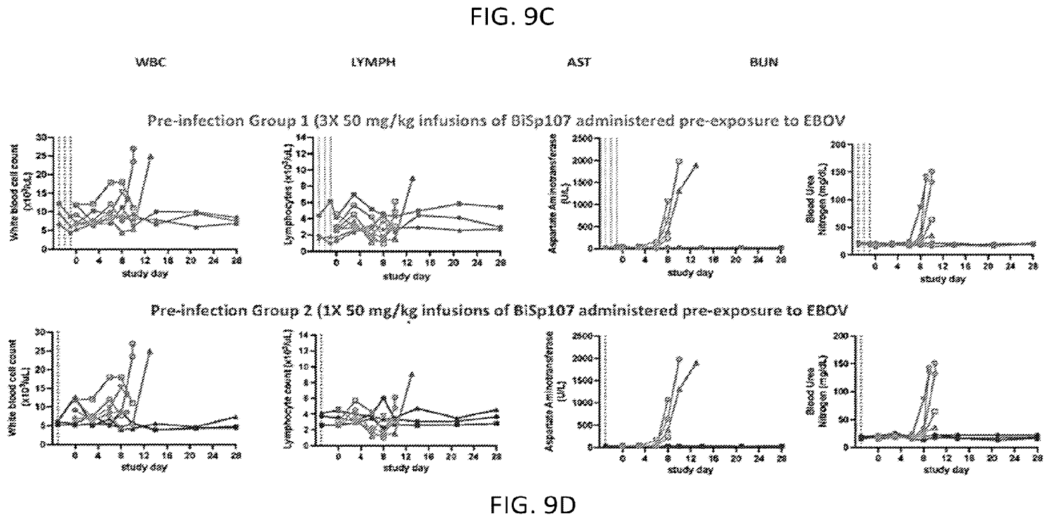

This example describes a study to determine the capacity of BiSp107 to protect rhesus macaques against lethal EBOV challenge. Macaques were exposed to a lethal dose of EBOV and administered BiSp107 at 50 mg/kg/day on days 1, 2 and 3 post-exposure (n=3) or on day 4 only (n=3) (FIG. 8A). All animals in the post-exposure treatment groups survived, whereas each of the control animals (n=5) succumbed to infection (FIG. 8B). Untreated animals displayed hallmark indicators of EBOV infection including increased alanine transaminase (ALT) and creatinine, decreased lymphocyte count and elevated viremia with 10⁶ to 10¹⁰ ge/ml (FIGS. 8C-8D, FIGS. 9A-9D). However, in contrast to previous experiments with mAb114 monotherapy given on days 1, 2 and 3 (Corti et al., Science 351: 1339-1342, 2016), transient viremia was not observed in any animals treated by BiSp107 (FIGS. 8C-8D). In addition, animals in the post-exposure treatment groups did not display any EBOV symptoms and were free of clinical markers indicative of EVD with normal lymphocyte count and blood chemistry measurements such as ALT and creatinine remaining within normal ranges (FIGS. 8C-8D). Together, these results illustrate the utility of BiSp107 as a treatment against EVD.

### Example 8: Pre-Infection Treatment by BiSp107

In an outbreak setting, following rVSVDG-ZEBOV-GP vaccination, “breakthrough” infections occur prior to developing full immunity and there are individuals who are at high-risk of exposure that are not candidates for vaccination (Mbala-Kingebeni et al., N Engl J Med 384(13):1240-1247, 2021). Thus, the ability to provide a monoclonal antibody to these individuals would be beneficial. Therefore, a study was performed to evaluate the capacity of BiSp107 to provide protection against lethal inoculation when given prior to infection as three daily doses of 50 mg/kg/dose starting on day −3 (n=3) or as a single infusion of 50 mg/kg on day −3 (n=3) (FIG. 8A). While each of the control animals succumbed to EVD, all pre-treated animals survived and did not show elevations in ALT, creatinine or signs of lymphopenia, markers consistent with EVD (FIGS. 8E-8F). These results demonstrate that pre-treatment with BiSp107 protects macaques from lethal infection and indicates that BiSp107 has clinical utility to prevent infection in high-risk individuals.

It will be apparent that the precise details of the methods or compositions described may be varied or modified without departing from the spirit of the described aspects of the disclosure. We chim al such modifications and variations that fall within the scope and spirit of the claims below.

1. A bispecific monoclonal antibody, comprising:
a first antigen binding portion comprising a heavy chain variable domain and a light chain variable domain, wherein the heavy chain variable domain comprises a heavy chain complementarity determining region (H-CDR)1, an H-CDR2 and an H-CDR3, and wherein the light chain variable domain comprises a light chain complementarity determining region (L-CDR)1, an L-CDR2 and an L-CDR3, wherein the H-CDR1, H-CDR2 and H-CDR3 are from the mAb114 heavy chain of SEQ ID NO: 2, and the L-CDR1, L-CDR2 and L-CDR3 are from the mAb114 light chain of SEQ ID NO: 4, and wherein the first antigen binding portion specifically binds to a first epitope of Ebola virus (EBOV) glycoprotein (GP); and
a second antigen binding portion comprising a heavy chain variable domain and a light chain variable domain, wherein the heavy chain variable domain comprises an H-CDR1, an H-CDR2 and an H-CDR3, and wherein the light chain variable domain comprises an L-CDR1, an L-CDR2 and an L-CDR3, wherein the H-CDR1, H-CDR2 and H-CDR3 are from the A09 heavy chain of SEQ ID NO: 6, and the L-CDR1, L-CDR2 and L-CDR3 are from the A09 light chain of SEQ ID NO: 8, and wherein the second antigen binding portion specifically binds a second epitope of EBOV GP.

2. The bispecific monoclonal antibody of claim 1, wherein:
the amino acid sequences of the H-CDR1, H-CDR2 and H-CDR3 of the first antigen binding portion respectively comprise SEQ ID NO: 12, SEQ ID NO: 13 and SEQ ID NO: 14; and/or
the amino acid sequences of the L-CDR1, L-CDR2 and L-CDR3 of the first antigen binding portion respectively comprise SEQ ID NO: 15, SEQ ID NO: 16 and SEQ ID NO: 17.

3. (canceled)

4. The bispecific monoclonal antibody of claim 1, wherein:
the amino acid sequences of the H-CDR1, H-CDR2 and H-CDR3 of the second antigen binding portion respectively comprise SEQ ID NO: 18, SEQ ID NO: 19 and SEQ ID NO: 20; and/or
the amino acid sequences of the L-CDR1, L-CDR2 and L-CDR3 of the second antigen binding portion respectively comprise SEQ ID NO: 21, SEQ ID NO: 22 and SEQ ID NO: 23.

5. (canceled)

6. The bispecific monoclonal antibody of claim 1, wherein:
the amino acid sequence of the heavy chain variable domain of the first antigen binding portion is at least 90% identical to residues 20-140 of SEQ ID NO: 2, and comprises the H-CDR1, H-CDR2 and H-CDR3 sequences of SEQ ID NO: 2;
the amino acid sequence of the light chain variable domain of the first antigen binding portion is at least 90% identical to residues 20-130 of SEQ ID NO: 4, and comprises the L-CDR1, L-CDR2 and L-CDR3 sequences of SEQ ID NO: 4;
the amino acid sequence of the heavy chain variable domain of the second antigen binding portion is at least 90% identical to residues 20-144 of SEQ ID NO: 6, and comprises the H-CDR1, H-CDR2 and H-CDR3 sequences of SEQ ID NO: 6; and/or
the amino acid sequence of the light chain variable domain of the second antigen binding portion is at least 90% identical to residues 20-124 of SEQ ID NO: 8, and comprises the L-CDR1, L-CDR2 and L-CDR3 sequences of SEQ ID NO: 8.

7. The bispecific monoclonal antibody of claim 1, wherein:
the amino acid sequence of the heavy chain variable domain of the first antigen binding portion comprises or consists of residues 20-140 of SEQ ID NO: 2;
the amino acid sequence of the light chain variable domain of the first antigen binding portion comprises or consists of residues 20-130 of SEQ ID NO: 4;
the amino acid sequence of the heavy chain variable domain of the second antigen binding portion comprises or consists of residues 20-144 of SEQ ID NO: 6; and/or
the amino acid sequence of the light chain variable domain of the second antigen binding portion comprises or consists of residues 20-124 of SEQ ID NO: 8.

8. The bispecific monoclonal antibody of claim 1, wherein the first antigen binding portion, the second antigen binding portion, or both, are a Fab fragment, a Fab′ fragment, a single chain Fv protein (scFv), or a disulfide stabilized Fv protein (dsFv).

9. The bispecific monoclonal antibody of claim 1, wherein:
the first antigen binding portion comprises a Fab comprising the heavy chain variable domain, the light chain variable domain, a heavy chain constant domain, and a light chain constant domain; and/or
the second antigen binding portion comprises a Fab comprising the heavy chain variable domain, the light chain variable domain, a heavy chain constant domain, and a light chain constant domain.

10. The bispecific monoclonal antibody of claim 9, wherein the heavy chain constant domain and the light chain constant domain of the first antigen binding portion are swapped.

11. The bispecific monoclonal antibody of claim 9, wherein:
the amino acid sequence of the heavy chain constant domain of the first antigen binding portion is at least 90% identical to residues 131-231 of SEQ ID NO: 4; and/or
the amino acid sequence of the light chain constant domain of the first antigen binding portion is at least 90% identical to residues 141-245 of SEQ ID NO: 2.

12. The bispecific monoclonal antibody of claim 9, wherein:
the amino acid sequence of the heavy chain constant domain of the first antigen binding portion comprises or consists of residues 131-231 of SEQ ID NO: 4; and/or
the amino acid sequence of the light chain constant domain of the first antigen binding portion comprises or consists of residues 141-245 of SEQ ID NO: 2.

13. (canceled)

14. The bispecific monoclonal antibody of claim 9, wherein:
the amino acid sequence of the heavy chain constant domain of the second antigen binding portion is at least 90% identical to residues 145-247 of SEQ ID NO: 6; and/or
the amino acid sequence of the light chain constant domain of the second antigen binding portion is at least 90% identical to residues 125-230 of SEQ ID NO: 8.

15. The bispecific monoclonal antibody of claim 14, wherein:
the amino acid sequence of the heavy chain constant domain of the second antigen binding portion comprises or consists of residues 145-147 of SEQ ID NO: 6; and/or
the amino acid sequence of the light chain constant domain of the second antigen binding portion comprises or consists of residues 125-230 of SEQ ID NO: 8.

16. The bispecific monoclonal antibody of claim 1, wherein:
the first antigen binding portion further comprises a first CH2 domain and a first CH3 domain; and
the second antigen binding portion further comprises a second CH2 domain and a second CH3 domain.

17. The bispecific monoclonal antibody of claim 16, wherein:
the amino acid sequence of the first CH2 domain and the first CH3 domain is at least 90% identical to residues 246-472 of SEQ ID NO: 2; and/or
the amino acid sequence of the second CH2 domain and the second CH3 domain is at least 90% identical to residues 248-474 of SEQ ID NO: 6.

18. The bispecific monoclonal antibody of claim 16, wherein:
the amino acid sequence of the first CH2 domain and the first CH3 domain comprises or consists of residues 246-472 of SEQ ID NO: 2; and/or
the amino acid sequence of the second CH2 domain and the second CH3 domain comprises or consists of residues 248-474 of SEQ ID NO: 6.

19. The bispecific monoclonal antibody of claim 1, wherein:
the first antigen binding portion comprises a heavy chain and a light chain, and the amino acid sequence of the heavy chain is at least 90% identical to residues 20-472 of SEQ ID NO: 2, and the amino acid sequence of the light chain is at least 90% identical to residues 20-131 of SEQ ID NO: 4; and/or
the second antigen binding portion comprises a heavy chain and a light chain, and the amino acid sequence of the heavy chain is at least 90% identical to residues 20-474 of SEQ ID NO: 6, and the amino acid sequence of the light chain is at least 90% identical to residues 20-230 of SEQ ID NO: 8.

20. The bispecific monoclonal antibody of claim 1, wherein:
the first antigen binding portion comprises a heavy chain and a light chain, and the amino acid sequence of the heavy chain comprises or consists of residues 20-472 of SEQ ID NO: 2, and the amino acid sequence of the light chain comprises or consists of residues 20-131 of SEQ ID NO: 4; and/or
the second antigen binding portion comprises a heavy chain and a light chain, and the amino acid sequence of the heavy chain comprises or consists of residues 20-474 of SEQ ID NO: 6, and the amino acid sequence of the light chain comprises or consists of residues 20-230 of SEQ ID NO: 8.

21. A composition comprising the bispecific monoclonal antibody of claim 1 and a pharmaceutically acceptable carrier.

22. A nucleic acid molecule encoding the bispecific monoclonal antibody of claim 1, or a heavy chain variable domain or a light chain variable domain thereof.

23. A nucleic acid molecule encoding a heavy chain or a light chain of a monoclonal antibody, wherein:
the nucleotide sequence of the heavy chain is at least 90% identical to SEQ ID NO: 1 or nucleotides 58-1422 of SEQ ID NO: 1;
the nucleotide sequence of the light chain is at least 90% identical to SEQ ID NO: 3 or nucleotides 58-699 of SEQ ID NO: 3;
the nucleotide sequence of the heavy chain is at least 90% identical to SEQ ID NO: 5 or nucleotides 58-1428 of SEQ ID NO: 5; or
the nucleotide sequence of the light chain is at least 90% identical to SEQ ID NO: 7 or nucleotides 58-702 of SEQ ID NO: 7.

24. The nucleic acid molecule of claim 23, wherein:
the nucleotide sequence of the heavy chain comprises or consists of SEQ ID NO: 1 or nucleotides 58-1422 of SEQ ID NO: 1;
the nucleotide sequence of the light chain comprises or consists of SEQ ID NO: 3 or nucleotides 58-699 of SEQ ID NO: 3;
the nucleotide sequence of the heavy chain comprises or consists of SEQ ID NO: 5 or nucleotides 58-1428 of SEQ ID NO: 5; or
the nucleotide sequence of the light chain comprises or consists of SEQ ID NO: 7 or nucleotides 58-702 of SEQ ID NO: 7.

25. The nucleic acid molecule of claim 22 operably linked to a promoter.

26. An expression vector comprising the nucleic acid molecule of claim 22.

27. A method of producing a bispecific monoclonal antibody that specifically binds Ebola virus glycoprotein, comprising:
transfecting host cells with a first expression vector comprising the nucleotide sequence of SEQ ID NO: 1, a second expression vector comprising the nucleotide sequence of SEQ ID NO: 3, a third expression vector comprising the nucleotide sequence of SEQ ID NO: 5 and a fourth expression vector comprising the nucleotide sequence of SEQ ID NO: 7; and
purifying the bispecific monoclonal antibody from the host cells and/or host cell culture supernatant.

28. A method of preventing, inhibiting or treating an Ebola virus (EBOV) infection in a subject, comprising administering to the subject a therapeutically effective amount of the bispecific monoclonal antibody of claim 1.

29. The method of claim 28, wherein the subject has an EBOV infection.

30. (canceled)

31. The method of claim 28, wherein the subject has been exposed to EBOV but has not been diagnosed as having an EBOV infection.

32. (canceled)

33. The method of claim 28, wherein the subject has not yet been exposed to EBOV.

34. (canceled)

35. The method of claim 28, wherein the bispecific monoclonal antibody is administered in a single dose or in multiple doses.

36-37. (canceled)

38. The method of claim 28, wherein administration of the bispecific monoclonal antibody reduces EBOV escape compared to administration of the combination of parental antibodies mAb114 and S1-4-A09.

39. (canceled)


SEQUENCE LISTING (23 sequences)

SEQ ID NO: 1 - DNA, 1422 bp, synthetic construct
  misc_feature 1..1422: note = Synthetic polynucleotide
  source 1..1422: mol_type = other DNA
atgggctggtcctgtattattctgttcctggtcgcaactgctactggggtccactccgaggtgcagctggtcgaaagcggggggggcctgatccagccaggaggctctctgaggctgagctgcgccgcctccggcttcgccctgaggatgtacgacatgcactgggtgaggcagaccatcgataagcgcctggagtgggtgtctgccgtgggacctagcggcgacacatactatgccgattccgtgaagggccggtttgccgtgagccgggagaacgccaagaatagcctgtccctgcagatgaacagcctgaccgccggcgacacagccatctactattgcgtgcggagcgataggggcgtcgcagggctgtttgattcttgggggcagggcattctggtgaccgtgagttcagctagcgtggcagcccctagcgtgttcatctttccccctagcgacgaacagctgaagagtggaactgcctccgtggtgtgcctgctgaataatttctatcctagggaggccaaggtccagtggaaagtggataacgctctgcagagcgggaattcccaggagtctgtgacagaacaggacagtaaggattcaacttatagcctgtctagtacactgactctgtccaaagcagactacgagaagcacaaagtctatgcctgcgaagtgactcatcaggggctgtcaagcccagtgaccaagtctttcaaccgcggagaatgtgacaaaacccacacatgcccaccatgtcctgcaccagagctgctgggaggaccaagcgtgttcctgtttcctccaaagcctaaagatactctgatgatctccagaacccccgaagtcacatgcgtggtcgtggacgtgtctcacgaggaccccgaagtcaagtttaactggtacgtggacggggtcgaggtgcataatgccaagacaaaaccccgagaggaacagtacaatagcacctatcgggtcgtgtccgtcctgacagtgctgcatcaggattggctgaacgggaaggagtataagtgtaaagtgagtaataaggcactgcccgcccctatcgagaaaaccattagcaaggctaaaggacagccaagagaaccccaggtgtgcacactgcccccttctagggacgagctgactaagaaccaggtctccctgtcttgtgctgtgaaaggcttctacccttcagatattgcagtggagtgggaaagcaatgggcagccagaaaacaattataagaccacaccacccgtgctggactccgatggatctttctttctggtctctaagctgaccgtggataaaagtcggtggcagcagggcaacgtctttagttgttcagtgatgcatgaggccctgcacaatcattacacacagaagagcctgtccctgtctcctggcaaatgatga

SEQ ID NO: 2 - protein, 472 aa, synthetic construct
  REGION 1..472: note = Synthetic polypeptide
MGWSCIILFLVATATGVHSEVQLVESGGGLIQPGGSLRLSCAASGFALRMYDMHWVRQTIDKRLEWVSAVGPSGDTYYADSVKGRFAVSRENAKNSLSLQMNSLTAGDTAIYYCVRSDRGVAGLFDSWGQGILVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 3 - DNA, 699 bp, synthetic construct
  misc_feature 1..699: note = Synthetic polynucleotide
  source 1..699: mol_type = other DNA
atgggctggtcctgtattatcctgttcctggtcgctaccgcaaccggcgtccactcagacattcagatgactcagtccccttcctccctgtctgccagcgtgggcgaccggatcaccatcacatgcagagccagccaggccttcgataactacgtggcctggtatcagcagaggcccggcaaggtgcctaagctgctgatctccgccgcatctgccctgcacgcaggagtgccatcccggttctccggatctggaagcggaacccactttaccctgacaatcagctccctgcagccagaggacgtggcaacatactattgtcagaactacaattccgcaccactgacatttgggggaggcactaaggtcgagattaagagcagcgcatcaaccaagggcccaagcgtgttcccactggctccctcaagcaagagtacttcaggaggaaccgctgcactgggatgtctggtgaaagattattttcccgaacctgtgaccgtcagctggaactccggcgccctgaccagcggagtgcacactttccctgctgtcctgcagtcaagcgggctgtattctctgtcttcagtggtcaccgtgccaagttcaagcctgggcacccagacatatatctgcaacgtcaatcataagccatccaatacaaaagtggataagaaagtcgaacccaaatcttgttgatga

SEQ ID NO: 4 - protein, 231 aa, synthetic construct
  REGION 1..231: note = Synthetic polypeptide
MGWSCIILFLVATATGVHSDIQMTQSPSSLSASVGDRITITCRASQAFDNYVAWYQQRPGKVPKLLISAASALHAGVPSRFSGSGSGTHFTLTISSLQPEDVATYYCQNYNSAPLTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 5 - DNA, 1428 bp, synthetic construct
  misc_feature 1..1428: note = Synthetic polynucleotide
  source 1..1428: mol_type = other DNA
atgggatggtcatgtatcatcctttttctagtagcaactgcaaccggtgtacattcccaggtccagctgcaggaaagcgggccaggactggtcaaaccctccgaaactctgtcactgacttgcgtggtctccggcggcattctgagctccttctactggaactggatcaggcagccacctggcaagggcctggagtggatcggcaatatctactattctggcagccccaactataatgcctcctttcagtctcgcgtggccatcagcgtggacacctccaagaaccagatctctctgaatctgaagagcgtgaccgccgccgatacagccatgtactattgcgtgcgggcatccagagcatacctgtggggctcttatcggccaaccgcactggatctgtgggggcagggctcactggtcactgtctcctcagcatcaacaaagggcccaagtgtgtttcccctggcaccttctagtaaatcaactagcggaggaaccgcagcactgggatgtctggtgaaggactatttccccgagcctgtcacagtgtcctggaactctggcgcactgaccagcggggtccacacatttccagccgtgctgcagtcaagcggcctgtacagcctgtcctctgtggtcactgtgcccagttcaagcctggggactcagacctatatctgcaacgtgaatcacaagccaagcaataccaaagtcgacaagaaagtggaacccaagtcctgtgataaaacacatacttgccctccatgtccagcaccagagctgctgggaggacctagcgtgttcctgtttccccctaagccaaaagataccctgatgatttctcggacacccgaagtcacttgcgtggtcgtggacgtcagtcacgaggaccccgaagtcaagttcaactggtacgtggacggggtcgaggtgcataatgctaagaccaaacccagggaggaacagtacaattcaacttatcgcgtcgtgagcgtcctgaccgtgctgcaccaggattggctgaacggaaaagagtacaagtgtaaagtgagcaataaggctctgcctgcaccaatcgagaaaaccattagcaaggccaaaggccagccaagagaaccccaggtgtacacactgccaccctgcagggatgagctgactaagaaccaggtcagcctgtggtgtctggtgaaagggttctatcctagtgacattgctgtggagtgggaatcaaatggacagccagaaaacaattacaagaccacacctccagtgctggacagtgatggatcattctttctgtattccaagctgacagtggacaaaagccggtggcagcagggcaacgtcttttcctgttctgtgatgcatgaggccctgcacaatcattacactcagaagagtctgtcactgagccctggcaaatgatga

SEQ ID NO: 6 - protein, 474 aa, synthetic construct
  REGION 1..474: note = Synthetic polypeptide
MGWSCIILFLVATATGVHSQVQLQESGPGLVKPSETLSLTCVVSGGILSSFYWNWIRQPPGKGLEWIGNIYYSGSPNYNASFQSRVAISVDTSKNQISLNLKSVTAADTAMYYCVRASRAYLWGSYRPTALDLWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 702 bp, synthetic construct
  misc_feature 1..702: note = Synthetic polynucleotide
  source 1..702: mol_type = other DNA
atgggatggtcatgtatcatcctttttctagtagcaactgcaaccggtgtccatgcttcctatgaactgacccagccccccagcgtgagcgtgagcccaggccagactgccaccattacttgtagcggagataaactgggcgacaagtacacaagctggttccagcagcggcccggacagtcccctctgctggtcatctaccaggataacaagcggccatctggcctgccagcacggttcagcggctccaactctggcaataccgccacactgaccatcagcggaacccaggcaatggacgaggcagattacctgtgtcaggtctgggatagcggggctgtcttcggagggggaacaaaactgactgtgctgggcgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 8 - protein, 230 aa, synthetic construct
  REGION 1..230: note = Synthetic polypeptide
MGWSCIILFLVATATGVHASYELTQPPSVSVSPGQTATITCSGDKLGDKYTSWFQQRPGQSPLLVIYQDNKRPSGLPARFSGSNSGNTATLTISGTQAMDEADYLCQVWDSGAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 9 - protein, 59 aa, synthetic construct
  REGION 1..59: note = Synthetic polypeptide
MKQIEDKIEEILSKIYHIENEIARIKKLIGEVASSSGLNDIFEAQKIEWHEAHHHHHHG

SEQ ID NO: 10 - DNA, 26 bp, synthetic construct
  misc_feature 1..26: note = Synthetic oligonucleotide
  source 1..26: mol_type = other DNA
atggtacctaaatgggcgttacagga

SEQ ID NO: 11 - DNA, 24 bp, synthetic construct
  misc_feature 1..24: note = Synthetic oligonucleotide
  source 1..24: mol_type = other DNA
cgacgcgttccaatacctgccggt

SEQ ID NO: 12 - protein, 8 aa, synthetic construct
GFALRMYD

SEQ ID NO: 13 - protein, 7 aa, synthetic construct
VGPSGDT

SEQ ID NO: 14 - protein, 13 aa, synthetic construct
VRSDRGVAGLFDS

SEQ ID NO: 15 - protein, 6 aa, synthetic construct
QAFDNY

SEQ ID NO: 16 - skipped

SEQ ID NO: 17 - protein, 9 aa, synthetic construct
QNYNSAPLT

SEQ ID NO: 18 - protein, 8 aa, synthetic construct
GGILSSFY

SEQ ID NO: 19 - protein, 7 aa, synthetic construct
IYYSGSP

SEQ ID NO: 20 - protein, 19 aa, synthetic construct
VRASRAYLWGSYRPTALDL

SEQ ID NO: 21 - protein, 6 aa, synthetic construct
KLGDKY

SEQ ID NO: 22 - skipped

SEQ ID NO: 23 - protein, 8 aa, synthetic construct
QVWDSGAV